(19) United States
(12) Patent Application Publication
(10) Pub. No.: US 2017/0143606 A1
(43) Pub. Date: May 25, 2017

(54) HAIR CARE AGENTS INCLUDING CASEIN HYDROLYSATE FOR IMPROVING HAIR STRUCTURE

(71) Applicant: Henkel AG & Co. KGaA, Duesseldorf (DE)

(72) Inventors: Melanie Giesen, Geldern (DE); Guido Fuhrmann, Erkelenz (DE); Sabine Gruedl, Erkelenz (DE); Waltraud Knieps-Massong, Korschenbroich (DE); Melanie Ludwig, Grevenbroich (DE)

(73) Assignee: Henkel AG & Co. KGaA, Duesseldorf (DE)

(21) Appl. No.: 15/356,518

(22) Filed: Nov. 18, 2016

(30) Foreign Application Priority Data
Nov. 20, 2015 (DE) 10 2015 222 976.9

(51) Int. Cl. A61K 8/64; A61Q 7/00; A61K 8/49; A61Q 5/00; A61K 8/97; A61K 8/44

(52) U.S. Cl. CPC A61K 8/64; A61K 8/97; A61K 8/44; A61K 8/4953; A61Q 5/00; A61Q 7/00

(57) ABSTRACT

Surprisingly, it has now been found that, when a hair care agent including at least one casein hydrolysate is applied, the hair growth itself and thus also the structure, elasticity, strength, resistance, and shine of the keratin fiber are significantly improved. Furthermore, is has surprisingly been found that the compositions according to the invention are suitable for triggering the release of growth factors and for strengthening and thickening the hair by stimulating the proliferation of the hair keratinocytes. The stimulation of the keratinocytes of the outer root sheath, which are partly responsible for the formation of the hair shaft, occurs by means of the growth factors HGF and KGF. Effects such as the “overconditioning” of the hair are avoided by means of the biologically based hair thickening. The hair grows more intensely from the root on and with a larger diameter, so this effect is particularly long-lasting.

### FIELD OF THE INVENTION

The present invention generally relates to hair care agents for improving hair structure.

### BACKGROUND OF THE INVENTION

In almost all cultures, the propagated and pursued ideal for the outward appearance of people comprises a full, well-groomed head of hair as an essential point. Therefore, premature hair loss is felt to be a flaw by most people. There have thus been many attempts to provide agents that counteract hair loss and stimulate increased or new hair growth. Therefore, there is a series of hair growth agents on the market, but the effectiveness thereof is disputed to say the least.

The reason for this poor state of affairs is that knowledge about the biological mechanism of hair growth is highly incomplete. There are only few empirical statements about modes of action, and there have been very few attempts at a general theory of hair growth.

In application EP 102534, it is described that carboxylic acids having an odd number of carbon atoms and a series of derivatives of said carboxylic acids are distinguished by a remarkably large hair-growth-stimulating effect.

In animal tests, K. Oba was able to demonstrate that, by applying pentadecanoic acid glycerol esters to the skin, energy is fed to the hair follicles.

Hair follicle cells are subject to a genetically defined cycle of growth, regression, and resting phase. The hair follicle is therefore the only organ that continually renews itself and thus has a unique metabolism that depends on the particular growth phase. The metabolism of the hair follicle comes almost completely to a standstill in the rest phase and is reinitiated with each new start of a further cycle.

This cycle is controlled by a small, highly specialized cell population in the hair bulb, the dermal papilla cells, which control the hair growth by means of a complex set of molecular signals that is specific to each phase of the hair cycle.

Human hair consists largely of keratins. Keratins are a group of fiber proteins that give elasticity and structure to the hair. These fiber cells surround the medulla, in the interior of which the medulla cells are located. The cortex makes up most (approximately 80%) of the hair shaft. The keratin fibers form the basis for the shine, elasticity, strength, and resistance of the hair.

EP 518192 B1 discloses a natural cosmetic skin or hair care agent including juices of pressed plants or plant parts, including lemon juice and olive oil, and water, characterized by a content of parsley juice, celery juice from leaves and tubers, dandelion juice from leaves, juice of peeled kiwis, yogurt, juice of pitted green olives, and sea salt. The agents disclosed here can also include yogurt.

EP 315541 B1 discloses a solid, preservative-free skin treatment agent based on yogurt or kefir, which skin treatment agent is mixed with water immediately before application and makes the skin smooth and soft.

GB 2,037,160 A discloses a product that is obtained from skim milk by protein hydrolysis under the conditions of yogurt production and can be used in cosmetic and pharmaceutical agents as a moisturizing agent.

In the laid-open European application EP 1813257 A, milk protein hydrolysate for the external care of the hair structure is disclosed. An effect on the hair root and support of the keratin synthesis are not disclosed.

In contrast to the treatment of the keratinized hair that has already grown out from the scalp, as described in the aforementioned prior art, the problem addressed by the present invention is that of positively influencing the biologically active part of the hair, the hair root. Increased keratin synthesis is able to fill up the hair structure again. Thus, the hair grows more intensely and with improved structure from the root out and thus provides a lasting possibility for improving the hair structure over the long term.

Surprisingly, it has now been found that, when a hair care agent including at least one casein hydrolysate is applied, the hair growth itself and thus also the structure, elasticity, strength, resistance, and shine of the keratin fiber are significantly improved.

Furthermore, is has surprisingly been found that the compositions according to the invention are suitable for triggering the release of growth factors and for strengthening and thickening the hair by stimulating the proliferation of the hair keratinocytes.

An exceptional influence of the agents according to the invention on the biological hair thickening was successfully detected. The stimulation of the keratinocytes of the outer root sheath, which are partly responsible for the formation of the hair shaft, occurs by means of the growth factors HGF and KGF. Effects such as the “overconditioning” of the hair are avoided by means of the biologically based hair thickening. The hair grows more intensely from the root on and with a larger diameter, so this effect is particularly long-lasting.

A further advantage of the present invention is that the agents according to the invention are able to positively influence the hair structure by stimulating the special hair-specific structural proteins (the hair keratins). Surprisingly, it was successfully shown that the gene expression of the hair keratins, hHa3-I and hHa4, is significantly increased. The hair structure, and thus the hair, is thereby strengthened. By influencing the hair structure already in the hair root, the hair can grow strongly and healthily without side effects such as the accumulation of care substances on the hair fiber.

Furthermore, it has been found that, by applying the agents according to the invention, the hair is positively influenced in its structure, growth, and metabolism. The gene expression of the hair genes important for this was significantly regulated by the use of the agent according to the invention. In particular, increased expression of genes which, in the extracellular matrix, are important for the anchoring of the hair in the scalp, was successfully observed. Both the dermal papilla and the entire bulb are surrounded by extracellular matrix, which anchors the hair follicle in the scalp. The support of the synthesis of extracellular matrix proteins such as laminin and collagen leads to particularly good anchoring of the hair in the scalp.

The penetration of active substances to the follicle can be hindered, because the target, the dermal papilla and the ORS keratinocytes, is embedded approximately 2 mm deep in the scalp. The use of liposomes can improve the penetration of an active substance, and therefore liposomally encapsulated compositions according to the invention can act very well. Surprisingly, it has been found that the compositions according to the invention themselves exhibit sufficient penetration to the location of action if the use of liposomes is not possible for reasons related to formulation.

Therefore, the present invention relates to a cosmetic method including a composition, which at least one casein hydrolysate and common carrier substances, active substances, and auxiliary substances for improving hair growth, hair structure, and the strength, resistance, and shine of the hair, and the cosmetic use of a composition including at least one casein hydrolysate and common carrier substances, active substances, and auxiliary substances for the care improvement of the hair growth, hair structure, and the strength, resistance, and shine of the hair.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic method for improving hair growth, hair structure, and the strength, resistance, and shine of the hair, characterized in that the hair is treated with a composition including at least one casein hydrolysate and common carrier substances, active substances, and auxiliary substances.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The hair treatment agents according to the invention for the method according to the invention and the use according to the invention include at least one decomposition product of casein. Casein is the most important protein fraction of (cow's) milk and is included therein at approximately 24 to 28 g/L. Casein makes up about 80% of the total protein of skim milk. Biochemically, a distinction must be made among αₛ₁-, αₛ₂-, β-, and κ-casein, which are synthesized in the cow at a molar ratio of approximately 8:2:8:3 (weight ratio of 38:10:36:13). The majority of the caseins exists in a state of association in casein micelles in milk. For each of the individual caseins, a large number of genetic variants is known.

All caseins are phosphoproteins; the phosphorus content is 0.9%. Because of a relatively high content of proline, caseins do not have tertiary structure—but have partial sequences of ordered secondary structure—are characterized by alternating series of hydrophobic and hydrophilic amino acid sequences, which gives them amphiphilic properties and enables the formation of micelles. Cross-links by means of thiol-disulfide exchange are possible only with αₛ₂-casein and κ-casein; αₛ₁- and β-casein do not include any cysteine/cystine.

αₛ₁-casein comprises 199 amino acid residues and approximately 8 to 10 phosphate residues and occurs in at least 5 genetic variants. αₛ₂-casein comprises 207 amino acid residues, including 2 cysteines. β-casein comprises 209 amino acid residues and is known in at least 7 genetic variants, which are generally phosphorylated five times in the N-terminal region. κ-casein comprises 169 amino acid residues, including 2 cysteines, and is known in 2 genetic variants.

In the context of the present invention, the term “casein hydrolysates” should be understood to mean decomposition products of caseins that are obtained by the acidic, alkaline, and/or enzymatic hydrolysis of the caseins themselves or of decomposition products thereof. The casein hydrolysates can be obtained from all types of milk, such as fresh milk, homogenized milk, pasteurized milk, quark, yogurt, kefir, cheese, or whey. All hydrolytically active enzymes, such as alkaline proteases, are suitable for the enzymatic decomposition. The decomposition is preferably performed down to even lower molar masses; hair treatment agents preferred according to the invention are characterized in that at least 50 mol %, preferably at least 70 mol %, and particularly at least 80 mol % of the casein hydrolysates included therein has a molar mass of 500 to 8000, preferably 800 to 8000, and particularly 1000 to 5000 daltons.

Especially preferred are casein hydrolysates produced from κ-casein and/or lactophorin. In a preferred embodiment of the invention, the composition in the method according to the invention or in the use according to the invention includes at least one casein hydrolysate in an amount of 0.0001 to 5 wt %, preferably 0.0005 to 5 wt %, especially preferably 0.001 wt % to 5 wt %, exceedingly preferably 0.005 wt % to 3.0 wt %, and extremely preferably 0.005 to 1 wt %, with respect to the total weight of the composition.

According to the invention, it is possible to add additional pharmacological and/or cosmetically active substances to the agents.

Examples of pharmacologically active substances are corticosteroids, β-blockers, estrogens, cyproterone acetate, and vasodilatory substances such as diazoxide, nifedipine, and minoxidil.

In addition to the aforementioned ingredients and optional additional ingredients, the agents according to the invention can include additional substances that prevent, alleviate, or cure hair loss. In particular, a content of hair-root-stabilizing active substances is advantageous. Said substances are described below:

Propecia (finasteride) is currently the only preparation which is approved worldwide and for which effectiveness and compatibility have been demonstrated in numerous studies. Propecia has the effect that less DHT can be formed from testosterone.

In addition to the casein hydrolysates according to the invention, L-carnitine derivatives can be used as additional especially preferred ingredients. The L-carnitine derivatives are selected particularly from acetyl-L-carnitine, L-carnitine fumarate, L-carnitine citrate, lauroyl-L-carnitine, and especially preferably L-carnitine tartrate. The mentioned L-carnitine compounds are available, for example, from Lonza GmbH (Wuppertal, Germany).

L-carnitine and/or L-carnitine derivatives are included in the agents according to the invention preferably in amounts of 0.001 to 10 wt %, with respect to the entire preparation. Amounts of 0.1 to 10 wt % are especially preferred, amounts of 0.1 to 5 wt % are particularly preferred, and amounts of 1 to 3 wt % are exceedingly preferred.

An additional preferred ingredient can be obtained from plants of the genus Echinacea. According to the invention, this is understood to mean the extract from the plant itself, the extract from the plant parts thereof, extracts and pressed juices of the coneflowers (Echinacea, synonym: Brauneria NECKER), particularly from Echinacea angustifolia DC, Echinacea paradoxa (NORTON), Echinacea simulata, E. atrorubens, E. tennesseensis, Echinacea strigosa (MCGREGOR), Echinacea laevigata, Echinacea purpurea (L.) Moench, and Echinacea pallida (Nutt), and active substances to be obtained from said extracts. Pressed juices and extracts of coneflowers, particularly of Echinacea purpurea (L.) MOENCH, are especially preferably used.

The pressed juices and/or extracts are preferably obtained from the stems and leaves (the above-ground plant parts) and/or roots of the coneflowers. The pressed juices are preferably obtained by mechanical pressing. Particularly preferred is pressing according to the method patented by the company Flachsmann as per EP 0730830 B1, the disclosure of which is hereby referenced in its entirety.

The extracts can be produced by means of water, polar or non-polar organic solvents, and mixtures thereof in the manner known to a person skilled in the art. Extracts that can be obtained by extraction by means of ethanol or water/ethanol mixtures, and pressed juice, are preferred.

The extracts in the original extracting agent as well as extracts/pressed juice in water or other organic solvents and/or a mixture thereof, particularly ethanol and ethanol/water mixtures, can be used. Extracted or pressed material is preferably used as a solid from which the solvent has been removed (particularly as gently as possible). However, also usable are extracts/pressed juices from which the solvent has been partially removed, so that a thickened extract/pressed juice is used. Pressed juices from the fresh Echinacea purpurea stems and leaves (Echinacea purpurea Moench herba) are exceedingly preferred. In particular, the extracts and/or pressed juices are used in solid form.

According to an especially preferred embodiment, the active substance that can be obtained from plants of the genus Echinacea is selected from pressed juices and extracts that can be obtained from Echinacea purpurea.

Active substances that can be obtained from plants of the genus Echinacea, preferably the pressed juices and/or extracts from Echinacea, are included in the agents according to the invention preferably in amounts of 0.001 to 10 wt %, with respect to the entire preparation. Amounts of 0.01 to 5 wt % are especially preferred, amounts of 0.01 to 5 wt % are particularly preferred, and amounts of 0.01 to 2 wt % are exceedingly preferred.

According to the invention, the compositions of the method according to the invention preferably also include taurine (2-aminoethanesulfonic acid), particularly N-alkyl derivatives of taurine. N-monomethyltaurine and N,N-dimethyltaurine are particularly suitable.

Taurine and/or derivatives thereof are included in the agents according to the invention preferably in amounts of 0.001 to 10 wt %, with respect to the entire preparation. Amounts of 0.05 to 5 wt % are especially preferred, amounts of 0.1 to 5 wt % are particularly preferred, and amounts of 0.5 to 3 wt % are exceedingly preferred.

Minoxidil, with or without supplemental additives, is probably the oldest demonstrably effective hair growth agent. To treat hair loss, it may be used only for external application. There are hair tonics that include 2%-5% of minoxidil and gels having up to 15% of minoxidil. The effectiveness increases with the dosage, but minoxidil is only soluble up to a content of 5% in hair tonics. In many countries, hair tonics having a minoxidil content of up to 2% are available without a prescription.

To combat the hormonal influences on the hair follicles, spironolactones can be used externally in the form of a hair tonic and in combination with minoxidil. Spironolactones act as androgen receptor blockers, i.e., the bonding of DHT to the hair follicles is prevented.

In summary, hair treatment agents according to the invention that additionally include—with respect to the weight thereof—0.001 to 5 wt % of hair-root-stabilizing substances, particularly minoxidil and/or finasteride and/or ketoconazole, are preferred.

According to the invention, O/W, W/O, and W/O/W emulsions in the form of creams or gels or surfactant-containing foaming solutions, such as shampoos, foam aerosols, or other preparations particularly suitable for use on the hair, are especially suitable according to the invention as cosmetic carriers. However, it is also conceivable that the ingredients are integrated into a powdery or tablet formulation, which is dissolved in water before use. In particular, the cosmetic carriers can be aqueous or aqueous-alcoholic.

An aqueous cosmetic carrier includes at least 50 wt % of water. In the sense of the present invention, the term “aqueous-alcoholic cosmetic carriers” should be understood to mean aqueous solutions including 3 to 70 wt % of a C₁-C₆ alcohol, particularly methanol, ethanol, or propanol, isopropanol, butanol, isobutanol, tert-butanol, n-pentanol, isopentanols, n-hexanol, isohexanols, propylene glycol, glycol, glycerol, 1,2-pentanediol, 1,5-pentanediol, 1,2-hexanediol, or 1,6-hexanediol. The agents according to the invention can additionally include additional organic solvents, such as methoxybutanol, benzyl alcohol, ethyl diglycol, or 1,2-propylene glycol. All water-soluble organic solvents are preferred.

In an embodiment preferred according to the invention, the composition includes, as a cosmetic carrier, at least 50 wt % of water and at least 0.1 to 20 wt %, preferably 0.1 to 10 wt %, especially preferably 0.1 to 5.0 wt %, exceedingly preferably 0.1 to 3.0 wt %, extremely preferably 0.1 to 1.0 wt % of a C₁-C₆ alcohol, particularly methanol, ethanol, or propanol, isopropanol, butanol, isobutanol, tert-butanol, n-pentanol, isopentanols, n-hexanol, isohexanols, glycol, propylene glycol, glycerol, 1,2-pentanediol, 1,5-pentanediol, 1,2-hexanediol, or 1,6-hexanediol. In this embodiment, the C₁-C₆ alcohol is especially preferably selected from ethanol, propylene glycol, glycerol, 1,2-pentanediol, 1,5-pentanediol, 1,2-hexanediol, or 1,6-hexanediol. In this embodiment, the C₁-C₆ alcohol is exceedingly preferably selected from propylene glycol and glycerol. Glycerol is extremely preferably used.

In summary, compositions according to the invention that contain, as a cosmetic carrier, at least 50 wt % of water and extremely preferably 0.1 to 1.0 wt % of glycerol are extremely preferred.

Of course, according to the invention, alcohols miscible with water only to a limited extent can also be used in the carrier.

“Miscible with water to a limited extent” is understood to mean alcohols that have a solubility in water at 20° C. of no more than 10 wt %, with respect to the water mass.

In many cases, triols and in particular diols have proven especially suitable according to the invention.

According to a further embodiment of the invention, alcohols having 4 to 20, particularly 4 to 10, carbon atoms can be used. The alcohols used can then be saturated or unsaturated and linear, branched, or cyclic. Examples are butanol-1, cyclohexanol, pentanol-1, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol and the Guerbet alcohols thereof, wherein this list should be understood to provide examples and to be unrestrictive. The compositions according to the invention for the cosmetic method according to the invention and for the use according to the invention including casein hydrolysate can be common skin and hair treatment agents.

The compositions can include all carrier substances, active substances, and auxiliary substances known and commonly used in these fields.

Such agents are, for example, shampoos, hair post-rinsing agents, hair gels, hair tonics, hair masks, hair creams, hair lotions, hair sprays, and hair tinctures. Said agents are typically used topically.

The compositions according to the invention for the cosmetic method according to the invention and the use according to the invention can exist in all formulations common for hair treatments, such as in the form of an aqueous solution or emulsions, such as an O/W or W/O emulsion, which can be produced in accordance with the phase inversion temperature method, a micro- or nanoemulsion, an aqueous-alcoholic or alcoholic solution, a cream, a gel, a lotion, or an aerosol. The compositions can comprise one or several phases.

Although the previously mentioned hair treatment agents are preferred according to the invention, the casein hydrolysates can also be added to other hair treatment agents, such as hair dyeing agents and perming agents. Said agents then optionally include the known direct dyes, precursors for oxidation dyes (developer and coupler components), and oxidants or reductants.

The compositions preferably have a pH value of 2 to 10, particularly 4 to 9.

In accordance with the type of hair treatment agent and the selected formulation type, the compositions for the cosmetic method according to the invention and the use according to the invention preferably include the following additional ingredients:

Fatty substances can additionally be used in the compositions as further skin and hair care substances. The term “fatty substances” should be understood to mean fatty acids, fatty alcohols, natural and synthetic waxes, which can be solid as well as liquid in aqueous dispersion, and natural and synthetic cosmetic oil components.

Linear and/or branched, saturated and/or unsaturated fatty acids having 6 to 30 carbon atoms can be used as fatty acids. Fatty acids having 10 to 22 carbon atoms are preferred. Notable among these are, for example, the isostearic acids such as the commercial products Emersol® 871 and Emersol® 875, isopalmitic acids such as the commercial product Edenor® IP 95, and all other fatty acids sold under the trade name Edenor® (Cognis). Additional typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid and technical mixtures thereof, which arise, for example, when natural fats and oils are subjected to high-pressure splitting, when aldehydes from Roelen oxo synthesis are reduced, or when unsaturated fatty acids are dimerized, Especially preferred are typically the fatty acid cuts that can be obtained from coconut oil or palm oil; the use of stearic acid is generally particularly preferred.

The usage amount is 0.1-15 wt %, with respect to the entire agent. The amount is preferably 0.1-10 wt %, wherein amounts of 0.1-5 wt % can be exceedingly advantageous.

Saturated, mono- or polyunsaturated, branched or unbranched fatty alcohols having C6-C30, preferably C10-C22, and exceedingly preferably C12-C22 carbon atoms can be used as fatty alcohols. For example, decanol, octanol, octenol, dodecenol, decenol, octadienol, dodecadienol, decadienol, oleyl alcohol, eruca alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol, and behenyl alcohol and the Guerbet alcohols thereof can be used according to the invention, wherein this list should be understood to provide examples and to be unrestrictive. However, the fatty alcohols are derived from preferably natural fatty acids, wherein it can usually be assumed that the fatty alcohols are obtained from the esters of the fatty acids by reduction. Fatty alcohol cuts which are produced by reducing naturally occurring triglycerides such as beef tallow, palm oil, peanut oil, colza oil, cottonseed oil, soy oil, sunflower oil, and linseed oil or fatty acid esters arising from the products of the transesterification of said triglycerides with corresponding alcohols, and which thus are a mixture of different fatty alcohols, are also usable according to the invention. Such substances can be purchased, for example, under the names Stenol®, e.g., Stenol® 1618, or Lanette®, e.g., Lanette® 0, or Lorol®, e.g., Lorol® C8, Lorol® C14, Lorol® C18, or Lorol® C8-18, HD-Ocenol®, Crodacol®, e.g., Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16, or Isocarb® 24. Of course, wool wax alcohols, which can be purchased, for example, under the names Corona®, White Swan®, Coronet®, or Fluilan®, can also be used according to the invention. The fatty alcohols are used in amounts of 0.1-30 wt %, with respect to the entire preparation, preferably in amounts of 0.1-20 wt %.

According to the invention, solid paraffins or isoparaffins, carnauba waxes, beeswaxes, candelilla waxes, ozokerites, ceresin, spermaceti, sunflower wax, fruit waxes such as apple wax or citrus wax, or microcrystalline waxes composed of PE or PP can be used as natural or synthetic waxes. Such waxes can be obtained, for example, from Kahl & Co., Trittau.

The usage amount is 0.1-50 wt % with respect to the entire agent, preferably 0.1-20 wt % and especially preferably 0.1-15 wt % with respect to the entire agent.

The natural and synthetic cosmetic oil bodies that can be advantageously used according to the invention include, for example:
plant oils. Examples of such oils are sunflower oil, olive oil, soy oil, rape oil, almond oil, jojoba oil, orange oil, wheat germ oil, peach kernel oil, and the liquid fractions of coconut oil. Other triglyceride oils, such as the liquid fractions of beef tallow and synthetic triglyceride oils, are also suitable.
liquid paraffin oils, isoparaffin oils, and synthetic hydrocarbons and di-n-alkyl ethers having a total of 12 to 36 C atoms, particularly 12 to 24 C atoms, such as di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, and di-tert-butyl ether, diisopentyl ether, di-3-ethyldecyl ether, tert-butyl n-octyl ether, isopentyl n-octyl ether, and 2-methylpentyl n-octyl ether. The compounds 1,3-di(2-ethylhexyl)-cyclohexane (Cetiol® S) and di-n-octyl ether (Cetiol® OE), which are available as commercial products, can be preferred.
ester oils. The term “ester oils” should be understood to mean the esters of C6-C30 fatty acids with C2-C30 fatty alcohols. The monoesters of the fatty acids with alcohols having 2 to 24 C atoms are preferred. Examples of fatty acid fractions used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid and technical mixtures thereof, which arise, for example, when natural fats and oils are subjected to high-pressure splitting, when aldehydes from Roelen oxo synthesis are reduced, or when unsaturated fatty acids are dimerized. Examples of the fatty alcohol fractions in the ester oils are isopropyl alcohol, caproic alcohol, capryl alcohol, 2-ethylhexanol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol, and brassidyl alcohol and technical mixtures thereof, which arise, for example, when technical methyl esters based on fats and oils or aldehydes from Roelen oxo synthesis are subjected to high-pressure hydrogenation and which arise as a monomer fraction when unsaturated fatty alcohols are dimerized. Especially preferred according to the invention are isopropyl myristate (Rilanit® IPM), isononaoic acid C16-18-alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate) (Cetiol®, lauric acid hexyl ester (Cetiol® A), di-n-butyl adipate (Cetiol® B), myristyl myristate (Cetiol® MM), cetearyl isononanoate (Cetiol® SN), and oleic acid decyl ester (Cetiol® V).
dicarboxylic acid esters such as di-n-butyl adipate, di(2-ethylhexyl) adipate, di(2-ethylhexyl) succinate, and diisotridecyl acelaate, and diol esters such as ethylene glycol dioleate, ethylene glycol diisotridecanoate, propylene glycol di(2-ethylhexanoate), propylene glycol diisostearate, propylene glycol dipelargonate, butanediol diisostearate, and neopentyl glycol dicaprylate,
symmetrical, unsymmetrical, or cyclic esters of carbonic acid with fatty alcohols, described, for example, in laid-open application DE 19756454, glycerol carbonate, or dicaprylyl carbonate (Cetiol® CC),
tri-fatty acid esters of saturated and/or unsaturated linear and/or branched fatty acids with glycerol,
fatty acid partial glycerides, i.e., monoglycerides, diglycerides, and technical mixtures thereof. If technical products are used, small amounts of triglycerides can still be included as a result of the manufacture. The partial glycerides preferably follow formula (D4-I),

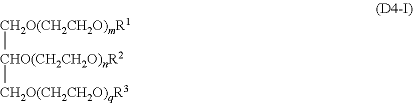

in which R¹, R², and R³ represent, independently of each other, hydrogen or a linear or branched, saturated and/or unsaturated acyl residue having 6 to 22, preferably 12 to 18 carbon atoms, with the stipulation that at least one of these groups represents an acyl residue and at least one of these groups represents hydrogen. The sum (m+n+q) represents 0 or numbers from 1 to 100, preferably 0 or 5 to 25. Preferably, R1 represents an acyl residue, R2 and R3 represent hydrogen, and the sum (m+n+q) is 0. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid, and erucic acid and technical mixtures thereof. Oleic acid monoglycerides are preferably used.

The usage amount of the natural and synthetic cosmetic oil bodies in the compositions for the cosmetic method according to the invention according to claim 1 and the use according to the invention according to claim 12 is typically 0.1 to 30 wt %, with respect to the entire composition, preferably 0.1 to 20 wt %, and particularly 0.1 to 15 wt %.

The total amount of oil components and fat components in the compositions for the cosmetic method according to the invention and the use according to the invention is typically 0.1-75 wt %, with respect to the entire composition. Amounts of 0.1-35 wt % are preferred according to the invention.

Surface-active compounds, particularly those from the group of the anionic, amphoteric, zwitterionic, and/or non-ionic surfactants, can be included in the agents as further constituents in addition to the casein hydrolysate according to the invention.

The term “surfactants” is understood to mean interface-active substances that can form adsorption layers at surfaces and interfaces or can aggregate to form association colloids or lyotropic mesophases in volume phases. A distinction is made between anionic surfactants consisting of a hydrophobic residue and a negatively charged hydrophilic headgroup, amphoteric surfactants, which bear both a negative charge and a compensating positive charge, cationic surfactants, which have a positively charged hydrophilic group in addition to a hydrophobic residue, and nonionic surfactants, which have no charges but instead have strong dipole moments and are strongly hydrated in aqueous solution.

All anionic surface-active substances suitable for use on the human body are suitable as anionic surfactants in the agents according to the invention. These are characterized by a water-solubilizing anionic group, such as a carboxylate, sulfate, sulfonate, or phosphate group, and a lipophilic alkyl group having approximately 8 to 30 C atoms. In addition, glycol or polyglycol ether groups, ester, ether, and amide groups, and hydroxyl groups can be included in the molecule. Examples of suitable anionic surfactants are, in each case in the form of the sodium, potassium, ammonium, and mono-, di-, and trialkanolammonium salts having 2 to 4 C atoms in the alkanol group,
linear and branched fatty acids having 8 to 30 C atoms (soaps),
ether carboxylic acids of the formula R—O—(CH2-CH2O)x CH2-COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of the formula R—O—(CH2-CH2O)x-OSO3H, in which R is a preferably linear alkyl group having 8 to 30 C atoms and x=0 or 1 to 12,
mixtures of surface-active hydroxysulfonates according to application DE 3725030,
sulfated hydroxyalkyl polyethylene glycol ethers and/or hydroxyalkylene propylene glycol ethers according to application DE 3723354,
sulfonates of unsaturated fatty acids having 8 to 24 C atoms and 1 to 6 double bonds according to application DE 3926344,
esters of tartaric acid and citric acid with alcohols that are products of the addition of approximately 2-15 molecules of ethylene oxide and/or propylene oxide to fatty alcohols having 8 to 22 C atoms,
alkyl and/or alkenyl ether phosphates of formula (E1-I),

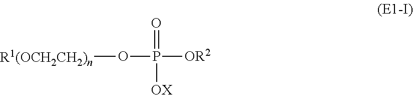

in which R1 preferably represents an aliphatic hydrocarbon residue having 8 to 30 carbon atoms, R2 represents hydrogen, a residue (CH2CH2O)nR1, or X, n represents numbers from 1 to 10, and X represents hydrogen, an alkali metal or alkaline-earth metal, or NR³R⁴R⁵R⁶, with R³ to R⁶ representing, independently of each other, hydrogen or a C1 to C4 hydrocarbon residue,
sulfated fatty acid alkylene glycol esters of formula (E1-II),

R⁷CO(AkO)ₙSO₃M (E1-II)
in which R⁷CO— represents a linear or branched, aliphatic, saturated and/or unsaturated acyl residue having 6 to 22 C atoms, Alk represents CH₂CH₂, CHCH₃CH₂, and/or CH₂CHCH₃, n represents numbers from 0.5 to 5, and M represents a cation, which are described in laid-open application DE 19736906.5,
monoglyceride sulfates and monoglyceride ether sulfates of formula (E1-III),

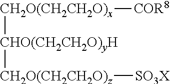

in which R⁸CO represents a linear or branched acyl residue having 6 to 22 carbon atoms, x, y, and z represent, in total, 0 or numbers from 1 to 30, preferably 2 to 10, and X represents an alkali metal or alkaline-earth metal. Typical examples of monoglyceride (ether) sulfates suitable according to the invention are the products of the reaction of lauric acid monoglyceride, coconut fatty acid monoglyceride, palmitic acid monoglyceride, stearic acid monoglyceride, oleic acid monoglyceride, and tallow fatty acid monoglyceride and ethylene oxide adducts thereof with sulfur trioxide or chlorosulfuric acid in the form of the sodium salts thereof. Monoglyceride sulfates of formula (E1-III), in which R8CO represents a linear acyl residue having 8 to 18 carbon atoms, which have been described, for example, in EP 0561825 B1, EP 0561999 B1, and DE 4204700 A1 or by A. K. Biswas et al. in J. Am. Oil Chem. Soc. 37, 171 (1960) and F. U. Ahmed in J. Am. Oil Chem. Soc. 67, 8 (1990), are preferably used.
amide ether carboxylic acids, which are described in EP 0690044,
condensation products of C8-C30 fatty alcohols with protein hydrolysates and/or amino acids and derivatives thereof, which are known to a person skilled in the art as protein fatty acid condensates, such as the Lamepon® types, the Gluadin® types, Hostapon® KCG, or the Amisoft® types.

Preferred anionic surfactants are alkyl sulfates, alkyl polyglycol ether sulfates, and ether carboxylic acids having 10 to 18 C atoms in the alkyl group and up to 12 glycol ether groups in the molecule, sulfosuccinic acid mono- and dialkyl esters having 8 to 18 C atoms in the alkyl group, and sulfosuccinic acid monoalkyl polyoxyethyl esters having 8 to 18 C atoms in the alkyl group and 1 to 6 oxyethyl groups, monoglyceride sulfates, alkyl and alkenyl ether phosphates, and protein fatty acid condensates.

Surface-active compounds that bear at least one quaternary ammonium group and at least one COO⁻ or —SO₃⁻ group in the molecule are referred to as zwitterionic surfactants. Especially suitable zwitterionic surfactants are the betaines such as the N-alkyl-N,N-dimethylammonium glycinates, for example coco alkyl dimethyl ammonium glycinate, N-acyl-aminopropyl-N,N-dimethylammonium glycinates, for example cocoacyl aminopropyl dimethyl ammonium glycinate, 2-alkyl-3-carboxymethyl-3-hydroxyethyl-imidazolines each having 8 to 18 C atoms in the alkyl or acyl group, and cocoacyl aminoethyl hydroxyethyl carboxymethyl glycinate. A preferred zwitterionic surfactant is the fatty acid amide derivative known under the INCI name Cocamidopropyl Betaine.

The term “ampholytic surfactants” is understood to mean surface-active compounds that include at least one free amino group and at least one —COOH or —SO₃H group in addition to a C8-C24 alkyl or acyl group in the molecule and are capable of forming inner salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids each having approximately 8 to 24 C atoms in the alkyl group. Especially preferred ampholytic surfactants are N-cocoalkylaminopropionate, cocoacylaminoethylaminopropionate, and C12-C18 acyl sarcosine.

Nonionic surfactants contain, for example, a polyol group, a polyalkylene glycol ether group, or a combination of polyol group and polyglycol ether group as a hydrophilic group. Such compounds are, for example,
products of the addition of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group,
products of the addition of 2 to 50 mol of ethylene oxide and/or 0 to 5 mol of propylene oxide to linear and branched fatty alcohols having 8 to 30 C atoms, to fatty acids having 8 to 30 C atoms, and to alkylphenols having 8 to 15 C atoms in the alkyl group, said products being end-capped with a methyl residue or C2-C6 alkyl residue, such as the types available under the sales names Dehydol® LS and Dehydol® LT (Cognis),
C12-C30 fatty acid mono- and diesters of products of the addition of 1 to 30 mol of ethylene oxide to glycerol,
products of the addition of 5 to 60 mol of ethylene oxide to castor oil and hardened castor oil,
polyol fatty acid esters, such as the commercial product Hydagen® HSP (Cognis) or Sovermol types (Cognis),
alkoxylated triglycerides,
alkoxylated fatty acid alkyl esters of formula (E4-I),

R¹CO—(OCH₂CHR²)_{w}OR³ (E4-I)
in which R¹CO represents a linear or branched, saturated and/or unsaturated acyl residue having 6 to 22 carbon atoms, R² represents hydrogen or methyl, R³ represents linear or branched alkyl residues having 1 to 4 carbon atoms, and w represents numbers from 1 to 20,
amine oxides,
hydroxy mixed ethers, which are described, for example, in laid-open application DE 19738866,
sorbitan fatty acid esters and products of the addition of ethylene oxide to sorbitan fatty acid esters, such as the polysorbates,
sugar fatty acid esters and products of the addition of ethylene oxide to sugar fatty acid esters,
products of the addition of ethylene oxide to fatty acid alkanolamides and fatty amines,
sugar surfactants of the type of the alkyl and alkenyl oligoglycosides according to formula (E4-II),

R⁴O-[G]ₚ (E4-II)
in which R⁴ represents an alkyl or alkenyl residue having 4 to 22 carbon atoms, G represents a sugar residue having 5 or 6 carbon atoms, and p represents numbers from 1 to 10. They can be obtained according to the relevant methods of preparative organic chemistry. As a representative of the extensive literature, reference is made here to the review by Biermann et al. in Starch/Starke 45, 281 (1993), B. Salka in Cosm. Toil. 108, 89 (1993), and J. Kahre et al. in SOFW Journal volume 8, 598 (1995).

The alkyl and alkenyl oligoglycosides can be derived from aldoses or ketoses having 5 or 6 carbon atoms, preferably from glucose. The preferred alkyl and/or alkenyl oligoglycosides are therefore alkyl and/or alkenyl oligoglucosides. The index number p in general formula (E4-II) indicates the degree of oligomerization, i.e., the distribution of mono- and oligoglycosides, and represents a number between 1 and 10. While p must always be an integer in the individual molecule, and can assume especially the values p=1 to 6 here, the value p for a certain alkyl oligoglycoside is an analytically determined calculated value, which is usually a rational number. Alkyl and/or alkenyl oligoglycosides having an average degree of oligomerization p of 1.1 to 3.0 are preferably used. Alkyl and/or alkenyl oligoglycosides having a degree of oligomerization of less than 1.7 and in particular between 1.2 and 1.4 are preferred from the perspective of application. The alkyl or alkenyl residue R4 can be derived from primary alcohols having 4 to 11, preferably 8 to 10 carbon atoms. Typical examples are butanol, caproic alcohol, capryl alcohol, capric alcohol, and undecyl alcohol, and technical mixtures thereof, which are obtained, for example, in the hydrogenation of technical fatty acid methyl esters or in the course of the hydrogenation of aldehydes from Roelen oxo synthesis. Alkyl oligoglucosides of chain length C8-C10 (degree of oligomerization=1 to 3) that arise as a forerun in the separation by distillation of technical C8-C18 coconut fatty alcohol and can be contaminated with a fraction of less than 6 wt % of C12 alcohol, and alkyl oligoglucosides based on technical C9/11 oxo alcohols (degree of oligomerization=1 to 3) are preferred. The alkyl or alkenyl residue R15 can also be derived from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol, and technical mixtures thereof, which can be obtained as described above. Alkyl oligoglucosides based on hardened C12/14 coconut alcohol having a degree of oligomerization of 1 to 3 are preferred.

Sugar surfactants of the type of the fatty acid N-alkyl polyhydroxyalkyl amides, a nonionic surfactant of formula (E4-III),

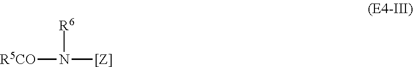

in which R⁵CO represents an aliphatic acyl residue having 6 to 22 carbon atoms, R⁶ represents hydrogen or an alkyl or hydroxyalkyl residue having 1 to 4 carbon atoms, and [Z] represents a linear or branched polyhydroxyalkyl residue having 3 to 12 carbon atoms and 3 to 10 hydroxyl groups. The fatty acid N-alkyl polyhydroxyalkyl amides are known substances that can typically be obtained by the reductive amination of a reducing sugar with ammonia, an alkylamine, or an alkanolamine and subsequent acylation with a fatty acid, a fatty acid alkyl ester, or a fatty acid chloride. In regard to the methods for the production thereof, reference is made to US patent documents U.S. Pat. No. 1,985,424, U.S. Pat. No. 2,016,962, and U.S. Pat. No. 2,703,798 and international patent application WO 92/06984. An overview of this topic by H. Kelkenberg can be found in Tens. Surf. Det. 25, 8 (1988). The fatty acid N-alkyl polyhydroxyalkyl amides are preferably derived from reducing sugars having 5 or 6 carbon atoms, particularly from glucose. The preferred fatty acid N-alkyl polyhydroxyalkyl amides are therefore fatty acid N-alkyl glucamides represented by the formula (E4-IV):

R⁷CONR⁸—CH₂—(CHOH)₄—CH₂OH (E4-IV)

Glucamides of formula (E4-IV), in which R⁸ represents hydrogen or an alkyl group and R⁷CO represents the acyl residue of caproic acid, caprylic acid, capric acid, lauric acid, myristic acid, palmitic acid, palmitoleic acid, stearic acid, isostearic acid, oleic acid, elaidic acid, petroselinic acid, linoleic acid, linolenic acid, arachidic acid, gadoleic acid, behenic acid, or erucic acid or technical mixtures thereof, are preferably used as fatty acid N-alkyl polyhydroxyalkyl amides. Especially preferred are fatty acid N-alkyl glucamides of formula (E4-IV) that are obtained by the reductive amination of glucose with methylamine and subsequent acylation with lauric acid or C12/14 coconut fatty acid or with a corresponding derivative. Furthermore, the polyhydroxyalkyl amides can also be derived from maltose and Palatinose.

The products of the addition of alkylene oxide to saturated linear fatty alcohols and fatty acids each having 2 to 30 moles of ethylene oxide per mole of fatty alcohol or fatty acid have proven to be preferred nonionic surfactants. Preparations having excellent properties are likewise obtained if they include fatty acid esters of ethoxylated glycerol as nonionic surfactants.

These compounds are characterized by the following parameters. The alkyl residue R includes 6 to 22 carbon atoms and can be both linear and branched. Primary linear aliphatic residues and primary aliphatic residues that are methyl-branched in the 2 position are preferred. Such alkyl residues are, for example, 1-octyl, 1-decyl, 1-lauryl, 1-myristyl, 1-cetyl, and 1-stearyl. 1-Octyl, 1-decyl, 1-lauryl, and 1-myristyl are especially preferred. If “oxo alcohols” are used as starting substances, compounds having an odd number of carbon atoms in the alkyl chain predominate.

Furthermore, the sugar surfactants are exceedingly preferred nonionic surfactants. These can be included in the compositions for the cosmetic method according to the invention according to claim 1 and the use according to the invention according to claim 12 preferably in amounts of 0.1-20 wt %, with respect to the entire composition. Amounts of 0.5-15 wt % are preferred. Amounts of 0.5-7.5 wt % are exceedingly preferred.

The compounds having alkyl groups used as a surfactant can be uniform substances in each case. However, it is generally preferred to proceed from virgin plant or animal raw materials in the production of these substances, substance mixtures having different alkyl chain lengths dependent on the particular raw material thus being obtained.

In the case of the surfactants that are products of the addition of ethylene oxide and/or propylene oxide to fatty alcohols or derivatives of these addition products, both products having a “normal” homolog distribution and products with a restricted homolog distribution can be used. The term “normal homolog distribution” is understood to mean mixtures of homologs that are obtained when fatty alcohol and alkylene oxide are reacted by using alkali metals, alkali metal hydroxides, or alkali metal alcoholates as catalysts. In contrast, restricted homolog distributions are obtained if, for example, hydrotalcites, alkaline-earth metal salts of ether carboxylic acids, alkaline-earth metal oxides, alkaline-earth metal hydroxides, or alkaline-earth metal alcoholates are used as catalysts. The use of products having a restricted homolog distribution can be preferred.

In addition to the mentioned components, the agents can include cationic surfactants of the type of the quaternary ammonium compounds, the esterquats, and the amidoamines as surface-active compounds. Preferred quaternary ammonium compounds are ammonium halides, particularly chlorides and bromides, such as alkyltrimethylammonium chlorides, dialkyldimethylammonium chlorides, and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride, and the imidazolium compounds known under the INCI names Quaternium-27 and Quaternium-83. The long alkyl chains of the aforementioned surfactants have preferably 10 to 18 carbon atoms.

Esterquats are known substances that include both at least one ester function and at least one quaternary ammonium group as a structural element. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. Such products are sold, for example, under the trademarks Stepantex®, Dehyquart®, and Armocare®. The products Armocare® VGH-70, an N,N-bis(2-palmitoyloxyethyl)dimethylammonium chloride, and Dehyquart® F-75, Dehyquart® C-4046, Dehyquart® L80, and Dehyquart® AU-35 are examples of such esterquats.

The alkylamidoamines are typically produced by the amidation of natural or synthetic fatty acids and fatty acid cuts with dialkylaminoamines. A compound from this substance group that is especially suitable according to the invention is the stearamidopropyl dimethylamine commercially available under the name Tegoamid® S 18.

The cationic surfactants are included in the compositions for the cosmetic method according to the invention and the use according to the invention preferably in amounts of 0.05 to 10 wt %, with respect to the entire composition. Amounts of 0.1 to 5 wt % are especially preferred.

In total, all surfactants are used in amounts of 0.1-50 wt %, preferably 0.5-30 wt %, and exceedingly preferably 0.5-25 wt %, with respect to the entire composition for the cosmetic method according to the invention and the use according to the invention.

In an additional preferred embodiment, emulsifiers are used in the compositions for the cosmetic method according to the invention and the use according to the invention. Emulsifiers cause the formation of water- or oil-stable adsorption layers at the phase interface, which adsorption layers protect the dispersed drops against coalescence and thus stabilize the emulsion. Therefore, like surfactants, emulsifiers are constructed of a hydrophobic molecule part and a hydrophilic molecule part. Hydrophilic emulsifiers preferably form O/W emulsions, and hydrophobic emulsifiers preferably form W/0 emulsions. The term “emulsion” should be understood to mean a distribution (dispersion), in the form of drops, of a liquid in another liquid achieved by applying energy in order to create stabilizing phase interfaces by means of surfactants. The selection of these emulsifying surfactants or emulsifiers is based on the substances to be dispersed and the outer phase and the fineness of the emulsion.

Emulsifiers that can be used according to the invention are, for example,
products of the addition of 4 to 30 mol of ethylene oxide and/or 0 to 5 mol of propylene oxide to linear fatty alcohols having 8 to 22 C atoms, to fatty acids having 12 to 22 C atoms, and to alkylphenols having 8 to 15 C atoms in the alkyl group,
C12-C22 fatty acid mono- and diesters of products of the addition of 1 to 30 mol of ethylene oxide to polyols having 3 to 6 carbon atoms, particularly to glycerol,
products of the addition of ethylene oxide and polyglycerol to methylglucoside fatty acid esters, to fatty acid alkanolamides, and to fatty acid glucamides,
C8-C22 alkyl mono- and oligoglycosides and ethoxylated analogs thereof, wherein degrees of oligomerization of 1.1 to 5, particularly 1.2 to 2.0, and glucose as a sugar component are preferred,
mixtures of alkyl (oligo)glucosides and fatty alcohols, for example the commercially available product Montanov® 68,
products of the addition of 5 to 60 mol of ethylene oxide to castor oil and hardened castor oil,
partial esters of polyols having 3-6 carbon atoms with saturated fatty acids having 8 to 22 C atoms,
sterols. The term “sterols” is understood to mean a group of steroids that bear a hydroxyl group at C atom 3 of the steroid skeleton and are isolated both from animal tissue (zoosterols) and from plant fats (phytosterols). Examples of zoosterols are cholesterol and lanosterol. Examples of suitable phytosterols are ergosterol, stigmasterol, and sitosterol. Sterols (the mycosterols) are also isolated from fungi and yeasts.
phospholipids. The term “phospholipids” is understood to mean, above all, the glucose phospholipids, which are obtained, for example, as lecithins or phosphatidylcholines from, for example, egg yolk or plant seeds (e.g., soybeans).
fatty acid esters of sugars and sugar alcohols, such as sorbitol,
polyglycerols and polyglycerol derivatives such as polyglycerol poly-12-hydroxystearate (commercial product Dehymuls® PGPH),
linear and branched fatty acids having 8 to 30 C atoms and the Na, K, ammonium, Ca, Mg, and Zn salts thereof

The compositions for the cosmetic method according to the invention and the use according to the invention include the emulsifiers preferably in amounts of 0.1-25 wt %, particularly 0.1-15 wt %, with respect to the entire composition.

The compositions for the cosmetic method according to the invention and the use according to the invention can preferably include at least one non-ionogenic emulsifier having an HLB of 8 to 18. Non-ionogenic emulsifiers having an HLB of 10-15 can be especially preferred according to the invention.

It has proven to be additionally advantageous that polymers can support the effect of the casein hydrolysate according to the invention. In a preferred embodiment, polymers are therefore added to the compositions for the cosmetic method according to the invention and the use according to the invention, wherein cationic, anionic, amphoteric, and non-ionic polymers have proven effective.

The term “cationic polymers” is understood to mean polymers that have a “temporarily” or “permanently” cationic group in the main chain and/or side chain. According to the invention, polymers that have a cationic group regardless of the pH value of the agent are called “permanently cationic”. They are generally polymers that include a quaternary nitrogen atom, for example in the form of an ammonium group. Preferred cationic groups are quaternary ammonium groups. In particular, polymers in which the quaternary ammonium groups are bonded to a polymer main chain constructed of acrylic acid, methacrylic acid, or derivatives thereof by means of a C1-4 hydrocarbon group have proven especially suitable.

Homopolymers of general formula (G1-I),

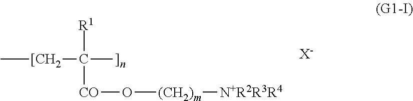

in which R¹═—H or —CH₃, R², R³, and R⁴ are selected independently of each other from C1-4 alkyl, alkenyl, or hydroxyalkyl groups, m=1, 2, 3, or 4, n is a natural number, and X— is a physiologically acceptable organic or inorganic anion, and copolymers consisting largely of the monomer units indicated in formula (G1-I) and non-ionogenic monomer units are especially preferred cationic polymers. In the context of these polymers, those for which at least one of the following conditions applies are preferred according to the invention:

R¹ represents a methyl group.

R², R³, and R⁴ represent methyl groups.

m has a value of 2.

Halide ions, sulfate ions, phosphate ions, methosulfate ions, and organic ions such as lactate, citrate, tartrate, and acetate ions, for example, are considered as physiologically acceptable counterions X—. Halide ions, particularly chloride, are preferred.

An especially suitable homopolymer is poly(methacryloyloxyethyl trimethylammonium chloride), which is cross-linked if desired and has the INCI name Polyquaternium-37. If desired, the cross-linking can be accomplished by means of polyolefinically unsaturated compounds, such as divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose, or glucose. Methylenebisacrylamide is a preferred cross-linking agent.

The homopolymer is preferably used in the form of a non-aqueous polymer dispersion, which should have a polymer fraction not below 30 wt %. Such polymer dispersions are commercially available under the names Salcare® SC 95 (approximately 50% polymer fraction, additional components: mineral oil (INCI name: Mineral Oil) and tridecyl polyoxypropylene polyoxyethylene ether (INCI name: PPG-1-Trideceth-6)) and Salcare® SC 96 (approximately 50% polymer fraction, additional components: mixture of diesters of propylene glycol with a mixture of caprylic acid and capric acid (INCI name: Propylene Glycol Dicaprylate/Dicaprate) and tridecyl polyoxypropylene polyoxyethylene ether (INCI name: PPG-1-Trideceth-6)).

Copolymers having monomer units according to formula (G1-I) preferably include acrylamide, methacrylamide, acrylic acid C1-4 alkyl esters, and methacrylic acid C1-4 alkyl esters as non-ionogenic monomer units. Among these non-ionogenic monomers, acrylamide is especially preferred. These copolymers, as in the case of the homopolymers described above, can be cross-linked. A copolymer preferred according to the invention is the cross-linked acrylamide/methacryloyloxyethyl trimethylammonium chloride copolymer. Such copolymers in which the monomers are present in a weight ratio of approximately 20:80 are commercially available as an approximately 50% non-aqueous polymer dispersion under the name Salcare® SC 92.

Additionally preferred cationic polymers are, for example:
quaternized cellulose derivatives, which are commercially available under the names Celquat® and Polymer JR®. The compounds Celquat® H 100, Celquat® L 200, and Polymer JR® 400 are preferred quaternized cellulose derivatives.
cationic alkyl polyglycosides according to patent document DE 4413686,
cationized honey, such as the commercial product Honeyquat® 50,
cationic guar derivatives, such as the products sold under the trade names Cosmedia® Guar and Jaguar® in particular,
polysiloxanes having quaternary groups, such as the commercially available products Q2-7224 (manufacturer: Dow Corning; a stabilized trimethylsilylamodimethicone), Dow Corning® 929 Emulsion (including a hydroxylamine-modified silicone, which is also referred to as amodimethicone), SM-2059 (manufacturer: General Electric), SLM-55067 (manufacturer: Wacker), and Abir-Quat 3270 and 3272 (manufacturer: Th. Goldschmidt; diquaternary polydimethylsiloxanes, Quaternium-80),
polymeric dimethyldiallylammonium salts and copolymers thereof with esters and amides of acrylic acid and methacrylic acid. The products commercially available under the names Merquat® 100 (poly(dimethyldiallylammonium chloride)) and Merquat® 550 (dimethyldiallylammoniumchloride/acrylamide copolymer) are examples of such cationic polymers.
copolymers of vinylpyrrolidone with quaternized derivatives of dialkylaminoalkyl acrylate and methacrylate, such as vinylpyrrolidone/dimethylaminoethyl methacrylate copolymers quaternized with diethyl sulfate. Such compounds are commercially available under the names Gafquat® 734 and Gafquat® 755.
vinylpyrrolidone/vinylimidazolium methochloride copolymers, which are offered under the names Luviquat® FC 370, FC 550, FC=905, and HM 552,
quaternized polyvinyl alcohol,
and the polymers having quaternary nitrogen atoms in the polymer main chain known under the names Polyquaternium 2, Polyquaternium 17, Polyquaternium 18, and Polyquaternium 27.

The polymers known under the names Polyquaternium-24 (commercial product, e.g., Quatrisoft® LM 200) likewise can be used as cationic polymers. The copolymers of vinylpyrrolidone, which are available as commercial products Copolymer 845 (manufacturer: ISP), Gaffix® VC 713 (manufacturer: ISP), Gafquat® ASCP 1011, Gafquat® HS 110, Luviquat® 8155, and Luviquat® MS 370, likewise can be used according to the invention.

Additional cationic polymers according to the invention are the “temporarily cationic” polymers. Said polymers typically include an amino group which, at certain pH values, exists as a quaternary ammonium group and thus is cationic. Preferred are, for example, chitosan and derivatives thereof, which are freely available commercially, for example under the trade names Hydagen® CMF, Hydagen® HCMF, Kytamer® PC, and Chitolam® NB/101.

Cationic polymers preferred according to the invention are cationic cellulose derivatives and chitosan and derivatives thereof, particularly the commercial products Polymer® JR 400, Hydagen® HCMF, and Kytamer® PC, cationic guar derivatives, cationic honey derivatives, particularly the commercial product Honeyquat® 50, cationic alkyl polyglycosides according to patent document DE 4413686, and polymers of the type Polyquaternium-37.

The anionic polymers that can be used with the casein hydrolysates in the agents according to the invention are anionic polymers that have carboxylate groups and/or sulfonate groups. Examples of anionic monomers of which such polymers can consist are acrylic acid, methacrylic acid, crotonic acid, maleic anhydride, and 2-acrylamido-2-methylpropane sulfonic acid. The acidic groups can be present completely or partially as a sodium, potassium, ammonium, or mono- or triethanolammonium salt. Preferred monomers are 2-acrylamido-2-methylpropane sulfonic acid and acrylic acid.

Anionic polymers that include 2-acrylamido-2-methylpropane sulfonic acid as the only monomer or as a comonomer, wherein the sulfonic acid group can be present completely or partially as a sodium, potassium, ammonium, or mono- or triethanolammonium salt, have proven exceedingly effective.

For example, such a homopolymer of 2-acrylamido-2-methylpropane sulfonic acid is commercially available under the name Rheothik® 11-80.

Within this embodiment, it can be preferred that copolymers consisting of at least one anionic monomer and at least one non-ionogenic monomer are used. With regard to the anionic monomers, reference is made to the substances indicated above. Preferred non-ionogenic monomers are acrylamide, methacrylamide, acrylic acid ester, methacrylic acid ester, vinylpyrrolidone, vinyl ether, and vinyl ester.

Preferred anionic copolymers are acrylic acid/acrylamide copolymers and, in particular, polyacrylamide copolymers having sulfonic-acid-group-containing monomers. An especially preferred anionic copolymer consists of 70 to 55 mol % of acrylamide and 30 to 45 mol % of 2-acrylamido-2-methylpropane sulfonic acid, wherein the sulfonic acid group can be present completely or partially as a sodium, potassium, ammonium, or mono- or triethanolammonium salt. This copolymer can also be cross-linked, wherein preferably polyolefinically unsaturated compounds such as tetraallyloxyethane, allyl sucrose, allyl pentaerythritol, and methylenebisacrylamide are used as cross-linking agents.

Likewise preferred anionic homopolymers are uncross-linked and cross-linked polyacrylic acids. Allyl ethers of pentaerythritol, of sucrose, and of propylene can be preferred cross-linking agents. Such compounds are commercially available, for example, under the trademark Carbopol®.

Copolymers of maleic anhydride and methyl vinyl ether, particularly those having cross-links, are likewise well suitable polymers. A maleic acid/methyl vinyl ether copolymer cross-linked by means of 1,9-decadiene is commercially available under the name Stabileze® QM.

Furthermore, amphoteric polymers can be used as polymers in all compositions for the cosmetic method according to the invention according to claim 1 and the use according to the invention according to claim 12. The term “amphoteric polymers” comprises polymers that include both free amino groups and free —COOH or SO₃H groups in the molecule and are capable of forming inner salts, zwitterionic polymers that include quaternary ammonium groups and —COO⁻ or —SO₃⁻ groups in the molecule, and polymers that include —COOH or SO₃H groups and quaternary ammonium groups.

An example of an amphopolymer that can be used according to the invention is the acrylic resin available under the name Amphomer®, which is a copolymer of tert-butylaminoethyl methacrylate, N-(1,1,3,3-tetramethylbutyl)acrylamide, and two or more monomers from the group of acrylic acid, methacrylic acid, and simple esters thereof

Additional amphoteric polymers that can be used according to the invention are the compounds mentioned in the British laid-open application GB 2104 091, the European laid-open application EP 47714, the European laid-open application EP 217274, the European laid-open application EP 283817, and the German laid-open application DE 2817369.

Amphoteric polymers that are preferably used are polymerizates that are composed largely of:

(a) monomers having quaternary ammonium groups of general formula (G34),

R¹—CH═CR²—CO—Z—(CₙH₂ₙ)—N⁽⁺⁾R³R⁴R⁵A⁽⁻⁾ (G3-I)
in which R¹ and R² represent, independently of each other, hydrogen or a methyl group and R³, R⁴, and R⁵ represent, independently of each other, alkyl groups having 1 to 4 carbon atoms, Z is an NH group or an oxygen atom, n is an integer from 2 to 5, and A⁽⁻⁾ is the anion of an organic or inorganic acid, and

(b) monomeric carboxylic acids of general formula (G3-II),

R⁶—CH═CR⁷—COOH (G3-II)
in which R⁶ and R⁷ are, independently of each other, hydrogen or methyl groups.

These compounds can be used both directly and in salt form, which is obtained by neutralizing the polymerizates, for example by means of an alkali metal hydroxide. With regard to the details of the manufacture of said polymerizates, reference is expressly made to the content of the German laid-open application DE 3929973. Exceedingly preferred are such polymerizates in which monomers of type (a) are used in which R³, R⁴, and R⁵ are methyl groups, Z is an NH group, and A⁽⁻⁾ is a halide, methoxysulfate, or ethoxysulfate ion; acrylamidopropyl trimethylammonium chloride is an especially preferred monomer (a). Acrylic acid is preferably used as monomer (b) for the mentioned polymerizates.

Furthermore, non-ionogenic polymers can be included in all compositions for the cosmetic method according to the invention and the use according to the invention.

Suitable non-ionogenic polymers are, for example:
Vinylpyrrolidone/vinyl ester copolymers, which are sold, for example, under the trademark Luviskol® (BASF). Luviskol® VA 64 and Luviskol® VA 73, both vinylpyrrolidone/vinyl acetate copolymers, are likewise preferred non-ionic polymers.
Cellulose ethers, such as hydroxypropyl cellulose, hydroxyethyl cellulose, and methylhydroxypropyl cellulose, which are sold, for example, under the trademarks Culminal® and Benecel® (AQUALON).
Shellac
Polyvinylpyrrolidones, which are sold, for example, under the name Luviskol® (BASF).
Siloxanes. Said siloxanes can be water-soluble or water-insoluble. Both volatile and non-volatile siloxanes are suitable, wherein compounds having a boiling point at normal pressure of greater than 200° C. are understood to be non-volatile siloxanes. Preferred siloxanes are polydialkylsiloxanes, such as polydimethylsiloxane, polyalkylarylsiloxanes, such as polyphenylmethylsiloxane, ethoxylated polydialkylsiloxanes, and polydialkylsiloxanes that include amine groups and/or hydroxy groups.
Gycosidically substituted silicones according to EP 0612759 B1.

It is also possible according to the invention that several, particularly two different polymers of the same charge and/or one ionic and one amphoteric and/or non-ionic polymer are included in the compositions for the cosmetic method according to the invention and the use according to the invention.

The polymers are included in the compositions for the cosmetic method according to the invention and the use according to the invention preferably in amounts of 0.01 to 10 wt %, with respect to the entire composition. Amounts of 0.1 to 5, particularly 0.1 to 3 wt %, are especially preferred.

Furthermore, protein hydrolysates and/or amino acids and derivatives thereof can be included in the compositions for the cosmetic method according to the invention and the use according to the invention. Said protein hydrolysates are, in each case, not identical to the casein hydrolysates according to the invention. Protein hydrolysates are product mixtures that are obtained by the acidically, basically, or enzymatically catalyzed decomposition of proteins. According to the invention, the term “protein hydrolysates” is understood to also mean total hydrolysates and individual amino acids and derivatives thereof and mixtures of different amino acids. According to the invention, the term “protein hydrolysates” is also understood to mean polymers constructed of amino acids and amino acid derivatives. Said polymers include, for example, polyalanine, polyasparagine, and polyserine. Additional examples of compounds that can be used according to the invention are L-alanyl-L-proline, polyglycine, glycyl-L-glutamine, or D/L-methionine-S-methyl sulfonium chloride. Of course, β amino acids and derivatives thereof, such as β-alanine, anthranilic acid, or hippuric acid, can also be used according to the invention. The molecular weight of the protein hydrolysates that can be used according to the invention is between 75, the molar weight of glycine, and 200000. The molar weight is preferably 75 to 50000, and exceedingly preferably 75 to 20000 daltons.

According to the invention, protein hydrolysates of plant origin or of animal or marine or synthetic origin can be used.

Animal protein hydrolysates are, for example, protein hydrolysates of elastin, collagen, keratin, silk, and milk protein, which can also be present in the form of salts. Such products are sold, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

The use of protein hydrolysates of plant origin, e.g., soy, almond, pea, potato, and wheat protein hydrolysates, is preferred according to the invention. Such products are available, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), and Crotein® (Croda).

The use of derivatives of the protein hydrolysates, for example in the form of the fatty acid condensation products thereof, is likewise possible. Such products are sold, for example, under the names Lamepon® (Cognis), Lexein® (Inolex), Crolastin® (Croda), and Crotein® (Croda).

According to the invention, it can be preferred that amino acids and/or oligopeptides are used as additional ingredients. In the present application, the term “amino acid” is also understood to mean a structure that includes only one permanently cationic group in the molecule, such as choline.

Amino acids according to the invention are selected from alanine, arginine, asparagine, aspartic acid, cysteine, cystine, citrulline, glutamic acid, glutamine, glycine, histidine, hydroxylysine, hydroxyproline, isoleucine, leucine, lysine, methionine, phenylalanine, proline, serine, threonine, thyroxine, tryptophan, tyrosine, acetyltyrosine, valine, betaine, ornithine, 1,1-dimethyl-proline, hercynine (Nα,Nα,Nα-trimethyl-L-histidinium betaine), ergothioneine (thioneine, 2-mercapto-Nα,Nα,Nα-trimethyl-L-histidinium betaine), and choline and mixtures thereof. According to the invention, all types of isomers, such as diastereomers, enantiomers, cis-trans isomers, optical isomers, conformational isomers, and racemates, can be used.

Alanine, arginine, asparagine, citrulline, glutamic acid, glutamine, glycine, histidine, hydroxylysine, hydroxyproline, isoleucine, leucine, lysine, proline, serine, betaine, ornithine, acetyltyrosine, 1,1-dimethyl-proline, choline, and mixtures thereof are especially preferably used.

Arginine, citrulline, glutamine, glycine, histidine, lysine, proline, serine, betaine, ornithine, acetyltyro sine, and mixtures thereof are exceedingly preferably used.

Arginine, citrulline, glutamine, glycine, histidine, lysine, acetyltyrosine, ornithine, and mixtures thereof are extremely preferably used.

In the sense of the present application, oligopeptides are condensation products of amino acids, which condensation products are linked by peptide bonds in the manner of an acid amide and comprise at least 3 and at most 25 amino acids. In hair treatment agents preferred according to the invention, the oligopeptide comprises 5 to 15 amino acids, preferably 6 to 13 amino acids, especially preferably 7 to 12 amino acids, and particularly 8, 9, or 10 amino acids.

An extremely preferred oligopeptide has the sequence Glu-Glu-Glu. The molar mass of the oligopeptide included in the agents according to the invention can vary depending on whether additional amino acids are bonded to the sequence Glu-Glu-Glu and depending on the type of said amino acids. Hair treatment agents preferred according to the invention are characterized in that the oligopeptide has a molar mass of 650 to 3000 daltons, preferably 750 to 2500 daltons, especially preferably 850 to 2000 daltons, and particularly 1000 to 1600 daltons.

An especially preferred oligopeptide additionally includes tyrosine, which is preferably bonded by means of the acid function thereof to the Glu-Glu-Glu sequence. Therefore, hair treatment agents preferred according to the invention are characterized in that the oligopeptide included therein has at least one amino acid sequence Tyr-Glu-Glu-Glu, wherein the amino group can be free or protonated and the carboxy groups can be free or deprotonated.

An additional especially preferred oligopeptide additionally includes isoleucine, which is preferably bonded by means of the amino function thereof to the Glu-Glu-Glu sequence. Therefore, hair treatment agents preferred according to the invention are characterized in that the oligopeptide included therein has at least one amino acid sequence Glu-Glu-Glu-Ile, wherein the amino group can be free or protonated and the carboxy groups can be free or deprotonated.

Oligopeptides that have both aforementioned amino acids (tyrosine and isoleucine) are preferred according to the invention. Especially preferred are hair treatment agents according to the invention in the case of which the oligopeptide included therein has at least one amino acid sequence Tyr-Glu-Glu-Glu-Ile, wherein the amino group can be free or protonated and the carboxy groups can be free or deprotonated.

An extremely preferred oligopeptide is commercially available from Croda under the trade name ProSina®.

The hair treatment agents according to the invention include the selected amino acids and/or the selected oligopeptides as previously described in a total amount—with respect to the entire agent—of 0.0001 to 10.0 wt %, especially preferably 0.0001 to 7.0 wt %, exceedingly preferably 0.0001 to 5.0 wt %.

The protein hydrolysates or derivatives thereof are included in the compositions for the cosmetic method according to the invention and the use according to the invention preferably in amounts of 0.1 to 10 wt %, with respect to the entire composition. Amounts of 0.1 to 5 wt % are especially preferred.

Furthermore, 2-pyrrolidinone-5-carboxylic acid and/or derivatives thereof can be used in the preparations of the method according to the invention. The sodium, potassium, calcium, magnesium, or ammonium salts in which the ammonium ion bears one to three C1 to C4 alkyl groups in addition to hydrogen are preferred. The sodium salt is exceedingly preferred. The amounts used in the agents according to the invention are 0.01 to 10 wt %, with respect to the entire agent, especially preferably 0.1 to 5, and particularly 0.1 to 3 wt %.

The use of vitamins, provitamins, and vitamin precursors and derivatives thereof has likewise proven advantageous.

According to the invention, vitamins, provitamins, and vitamin precursors that are typically assigned to the groups A, B, C, E, F, and H are preferred.

Retinol (vitamin A1) and 3,4-didehydroretinol (vitamin A2) belong to the group of substances referred to as vitamin A. β-carotene is the provitamin of retinol. For example, vitamin A acid and esters thereof, vitamin A aldehyde, and vitamin A alcohol and esters thereof such as the palmitate and the acetate are considered as a vitamin A component according to the invention. The preparations used according to the invention include the vitamin A component preferably in amounts of 0.05-1 wt %, with respect to the entire preparation.

The vitamin B group or the vitamin B complex includes, among other things:
Vitamin B1 (thiamine)
Vitamin B2 (riboflavin)
Vitamin B3. This designation is often used to refer to the compounds nicotinic acid and nicotinic acid amide (niacinamide). Preferred according to the invention is nicotinic acid amide, which is included in the agents used according to the invention preferably in amounts of 0.05 to 1 wt %, with respect to the entire agent.
Vitamin B5 (pantothenic acid, panthenol, and pantolactone). In the context of this group, panthenol and/or pantolactone is preferably used. Derivatives of panthenol that can be used according to the invention are, in particular, the esters and ethers of panthenol and cationically derivatized panthenols. Individual representatives are, for example, panthenol triacetate, panthenol monoethyl ether and the monoacetate thereof, and the cationic panthenol derivatives disclosed in WO 92/13829. The mentioned compounds of the vitamin B5 type are included in the agents used according to the invention preferably in amounts of 0.05-10 wt %, with respect to the entire agent. Amounts of 0.1-5 wt % are especially preferred.
Vitamin B6 (pyridoxine, pyridoxamine, and pyridoxal).

Vitamin C (ascorbic acid). Vitamin C is used in the agents used according to the invention preferably in amounts of 0.1 to 3 wt %, with respect to the entire agent. Use in the form of palmitic acid ester, glucosides, or phosphates can be preferred. Use in combination with tocopherols can likewise be preferred.

Vitamin E (tocopherols, particularly α-tocopherol). Tocopherol and derivatives thereof, under which, in particular, the esters and the acetate, the nicotinate, the phosphate, and the succinate fall, are included in the compositions for the cosmetic method according to the invention and the use according to the invention preferably in amounts of 0.05-1 wt %, with respect to the entire composition.

Vitamin F. The term “vitamin F” is typically understood to mean essential fatty acids, particularly linoleic acid, linolenic acid, and arachidonic acid.

Vitamin H. The compound (3aS,4S,6aR)-2-oxohexahydrothienol[3,4-d]imidazole-4-valeric acid is referred to as vitamin H, but in the meantime the trivial name biotin has become established for said compound. Biotin is included in the compositions for the cosmetic method according to the invention and the use according to the invention preferably in amounts of 0.0001 to 1.0 wt %, particularly in amounts of 0.001 to 0.01 wt %.

The preparations used according to the invention preferably include vitamins, provitamins, and vitamin precursor from groups A, B, E, and H. Of course, several vitamins and vitamin precursors can also be included simultaneously.

Panthenol, pantolactone, pyridoxine and derivatives thereof, nicotinic acid amide, and biotin are especially preferred. The usage amount of vitamins and vitamin precursors in the compositions for the cosmetic method according to the invention and the use according to the invention is typically 0.0001-10 wt %, with respect to the entire composition, preferably 0.0001-5 wt %, and particularly 0.0001-3 wt %.

Finally, plant extracts can be used in the compositions for the cosmetic method according to the invention and the use according to the invention.

These extracts are typically produced by extraction of the entire plant. However, in individual cases, it can also be preferred that the extracts are produced exclusively from flowers and/or leaves of the plant.

In particular, the extracts from green tea, oak bark, nettles, witch hazel, hops, henna, chamomile, burdock root, horsetail, whitethorn, linden blossoms, almond, aloe vera, spruce needle, Aesculus, sandalwood, juniper, coconut, mango, apricot, lime, wheat, kiwi, melon, orange, grapefruit, sage, rosemary, birch, mallow, meadowfoam, wild thyme, yarrow, garden thyme, melissa, restharrow, coltsfoot, marshmallow, meristem, ginseng, and ginger root are preferred according to the invention.

The extracts from green tea, oak bark, nettles, witch hazel, hops, chamomile, burdock root, horsetail, linden blossoms, almond, aloe vera, coconut, mango, apricot, lime, wheat, kiwi, melon, orange, grapefruit, sage, rosemary, birch, meadowfoam, wild thyme, yarrow, restharrow, meristem, ginseng, and ginger root are especially preferred.

The extracts from green tea, almond, aloe vera, coconut, mango, apricot, lime, wheat, kiwi, and melon are exceedingly suitable for the use according to the invention. The Echinacea extract described at the beginning is an extremely preferred extract.

Water, alcohols, and mixtures thereof can be used as extracting agents for producing the mentioned plant extracts. Among the alcohols, lower alcohols such as ethanol and isopropanol, but in particular polyhydric alcohols such as ethylene glycol and propylene glycol, both as a sole extracting agent and in mixture with water, are preferred. Plant extracts based on water/propylene glycol at a ratio of 1:10 to 10:1 have proven especially suitable.

According to the invention, the plant extracts can be used in pure form or in diluted form. If the plant extracts are used in diluted form, the plant extracts typically include approximately 2-80 wt % of active substance and, as a solvent, the extracting agent or extracting agent mixture used in obtaining the active substance.

Furthermore, it can be preferred that mixtures of several, particularly two, different plant extracts are used in the agents according to the invention.

The usage amount of the plant extracts in the compositions for the cosmetic method according to the invention and the use according to the invention is typically 0.01-50 wt %, with respect to the entire composition, preferably 0.1-30 wt %, and particularly 0.1-20 wt %.

Advantageously according to the invention, short-chain carboxylic acids can additionally be used. According to the invention, short-chain carboxylic acids and derivatives thereof are understood to mean carboxylic acids that can be saturated or unsaturated and/or straight-chain or branched or cyclic and/or aromatic and/or heterocyclic and have a molecular weight of less than 750. Saturated or unsaturated straight-chain or branched carboxylic acids having a chain length of 1 to 16 C atoms in the chain can be preferred according to the invention. Such carboxylic acids having a chain length of 1 to 12 C atoms in the chain are exceedingly preferred.

The short-chain carboxylic acids according to the invention can have one, two, three, or more carboxy groups. Carboxylic acids having several carboxy groups, particularly di- and tricarboxylic acids, are preferred according to the invention. The carboxy groups can be present completely or partially as an ester, acid anhydride, lactone, amide, imidic acid, lactam, lactim, dicarboximide, carbohydrazide, hydrazone, hydroxam, hydroxime, amidine, amide oxime, nitrile, phosphon, or phosphate ester. Of course, the carboxylic acids according to the invention can be substituted along the carbon chain or the ring skeleton. The substituents of the carboxylic acids according to the invention should include, for example, C1-C8 alkyl, C2-C8 alkenyl, aryl, aralkyl, and aralkenyl, hydroxymethyl, C2-C8 hydroxyalkyl, C2-C8 hydroxyalkenyl, aminomethyl, C2-C8 aminoalkyl, cyano, formyl, oxo, thioxo, hydroxy, mercapto, amino, carboxy, or imino groups. Preferred substituents are C1-C8 alkyl, hydroxymethyl, hydroxy, amino, and carboxy groups. Especially preferred are substituents in the a position. Exceedingly preferred substituents are hydroxy, alkoxy, and amino groups, wherein the amino function can be optionally further substituted by alkyl, aryl, aralkyl, and/or alkenyl residues. Furthermore, the phosphonic and phosphate esters are likewise preferred carboxylic acid derivatives.

The following are mentioned as examples of carboxylic acids according to the invention: formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o-, m-, p-phthalic acid, naphthoic acid, toluic acid, hydratropic acid, atropic acid, cinnamic acid, isonicotinic acid, nicotinic acid, bicarbamic acid, 4,4′-dicyano-6,6′-binicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamic acid, 1-pyrazolecarboxylic acid, gallic acid, or propanetricarboxylic acid, a dicarboxylic acid selected from the group formed by compounds of general formula (N-I),

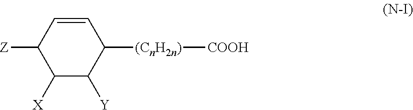

in which Z represents a linear or branched alkyl or alkenyl group having 4 to 12 carbon atoms, n represents a number from 4 to 12, and one of the two groups X and Y represents a COOH group and the other represents hydrogen or a methyl or ethyl residue, dicarboxylic acids of general formula (N-I) that additionally bear 1 to 3 methyl or ethyl sub stituents on the cyclohexene ring, and dicarboxylic acids that technically arise from the dicarboxylic acids according to formula (N-I) by the addition of a molecule of water to the double bond in the cyclohexene ring.

Such compounds are commercially available under the names Westvaco Diacid® 1550 and Westvaco Diacid® 1595 (manufacturer: Westvaco).

In addition to the short-chain carboxylic acids according to the invention listed above as examples, physiologically acceptable salts thereof can also be used according to the invention. Examples of such salts are the alkali, alkaline-earth, zinc, and ammonium salts, by which the mono-, di-, and trimethyl-, -ethyl-, and -hydroxyethylammonium salts should also be understood in the context of the present application. However, acids neutralized with basically reacting amino acids, such as arginine, lysine, ornithine, and histidine, can be exceedingly preferably used according to the invention. Furthermore, for formulation reasons, it can be preferred that the carboxylic acid is selected from the water-soluble representatives, particularly the water-soluble salts.

Furthermore, it is preferred according to the invention that hydroxy carboxylic acids and in particular the dihydroxy, trihydroxy, and polyhydroxy carboxylic acids and the dihydroxy, trihydroxy, and polyhydroxy di-, tri-, and polycarboxylic acids are used.

Preferred hydroxy carboxylic acid esters are, for example, full esters of glycolic acid, lactic acid, malic acid, tartaric acid, or citric acid. Additional fundamentally suitable hydroxy carboxylic acid esters are esters of β-hydroxypropionic acid, tartronic acid, D-gluconic acid, sugar acid, mucic acid, or glucuronic acid. Primary, linear or branched aliphatic alcohols having 8-22 C atoms, such as fatty alcohols or synthetic fatty alcohols, are suitable as alcohol components of these esters. The esters of C12-C15 fatty alcohols are especially preferred. Esters of this type are commercially available, for example, under the trademark Cosmacol® of EniChem, Augusta Industriale. Especially preferred polyhydroxy polycarboxylic acids are polylactic acid and polytartaric acid and esters thereof

Heterocyclic compounds, such as imidazole, pyrrolidine, piperidine, dioxolane, dioxan, morpholine, and piperazine can be used as additional ingredients that support the effect of the casein hydrolysate. Furthermore, derivatives of these compounds, such as the C1-4 alkyl derivatives, C1-4 hydroxyalkyl derivatives, and C1-4 aminoalkyl derivatives, are suitable. Preferred substituents, which can be positioned on carbon atoms or nitrogen atoms of the heterocyclic ring systems, are methyl, ethyl, β-hydroxyethyl, and β-aminoethyl groups. These derivatives preferably include 1 or 2 of these substituents.

Derivatives of heterocyclic compounds preferred according to the invention are, for example, 1-methylimidazole, 2-methylimidazole, 4(5)-methylimidazole, 1,2-dimethylimidazole, 2-ethylimidazole, 2-isopropylimidazole, N-methylpyrrolidone, 1-methylpiperidine, 4-methylpiperidine, 2-ethylpiperidine, 4-methylmorpholine, 4-(2-hydroxyethyl)morpholine, 1-ethylpiperazine, 1-(2-hydroxyethyl)piperazine, 1-(2-aminoethyl)piperazine. Additional imidazole derivatives preferred according to the invention are biotin, hydantoin, and benzimidazole.

Among these heterocyclic care substances, the mono- and dialkylimidazoles, biotin, hydantoin, and in particular imidazole itself are especially preferred.

These heterocyclic compounds are included in the compositions for the cosmetic method according to the invention and the use according to the invention in amounts of 0.5 to 10 wt %, with respect to the entire composition. Amounts of 2 to 6 wt % have proven especially suitable.

The agents according to the invention can include at least one carbohydrate from the group of the monosaccharides, disaccharides, and/or oligosaccharides as an additional constituent. Here, hair treatment agents preferred according to the invention are characterized in that they include—with respect to the weight thereof—0.01 to 5 wt %, preferably 0.05 to 4.5 wt %, especially preferably 0.1 to 4 wt %, more preferably 0.5 to 3.5 wt %, and particularly 0.75 to 2.5 wt %, of carbohydrate(s), selected from monosaccharides, disaccharides, and/or oligosaccharides, as a care substance, wherein preferred carbohydrates are selected from
monosaccharides, particularly D-ribose and/or D-xylose and/or L-arabinose and/or D-glucose and/or D-mannose and/or D-galactose and/or D-fructose and/or sorbose and/or L-fucose and/or L-rhamnose,
disaccharides, particularly saccharose and/or maltose and/or lactose and/or trehalose and/or cellobiose and/or gentiobiose and/or isomaltose.

In an additional embodiment preferred according to the invention, the compositions according to the invention include bioquinones. In the agents according to the invention, suitable bioquinones are one or more ubiquinones and/or plastoquinones. The ubiquinones preferred according to the invention have the following formula:

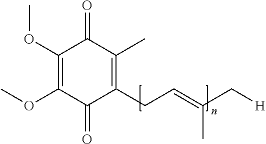

with n=6, 7, 8, 9, or 10.

The coenzyme Q₁₀ is most preferred.

Preferred compositions according to the invention include purine and/or purine derivatives in narrow amount ranges. Here, cosmetic agents preferred according to the invention are characterized in that they include—with respect to the weight thereof—0.001 to 2.5 wt %, preferably 0.0025 to 1 wt %, especially preferably 0.005 to 0.5 wt %, and particularly 0.01 to 0.1 wt % of purine(s) and/or purine derivative(s). Cosmetic agents preferred according to the invention are characterized in that they include purine, adenine, guanine, uric acid, hypoxanthine, 6-purinethiol, 6-thioguanine, xanthine, caffeine, theobromine, or theophylline. In hair cosmetic preparations, caffeine is most preferred.

In an additional preferred embodiment of the present invention, the cosmetic agent includes ectoine ((S)-2-methyl-1,4,5,6-tetrahydro-4-pyrimidinecarboxylic acid).

In addition, it can also prove advantageous if the compositions for the cosmetic method according to the invention and the use according to the invention include penetration enhancers and/or swelling agents. For example, urea and urea derivatives, guanidine and derivatives thereof, arginine and derivatives thereof, water glass, imidazole and derivatives thereof, histidine and derivatives thereof, benzyl alcohol, glycerol, glycol and glycol ether, propylene glycol and propylene glycol ethers, for example propylene glycol monoethyl ether, carbonates, hydrogen carbonates, diols and triols, and particularly 1,2-diols and 1,3-diols, such as 1,2-propanediol, 1,2-pentanediol, 1,2-hexanediol, 1,2-dodecanediol, 1,3-propanediol, 1,6-hexanediol, 1,5-pentanediol, and 1,4-butanediol, should be regarded as penetration enhancers and/or swelling agents. The penetration enhancers and swelling agents are included in the preparations used according to the invention in amounts of 0.1 to 20 wt %, with respect to the entire agent. Amounts of 01 to 10 wt % are preferred.

Furthermore, silicone oils and silicone gums, particularly dialkyl- and alkylarylsiloxanes, such as dimethylpolysiloxane and methylphenylpolysiloxane, and alkoxylated and quaternized analogs thereof, are suitable as conditioning active substances. Examples of such silicones are the products sold by Dow Corning under the names DC 190, DC 200, and DC 1401 and the commercial product Fancorsil® LIM-1.

Likewise suitable as conditioning active substances according to the invention are cationic silicone oils such as the commercially available products Q2-7224 (manufacturer: Dow Corning; a stabilized trimethylsilylamodimethicone), Dow Corning® 939 Emulsion (containing a hydroxylamine-modified silicone, which is also referred to as amodimethicone), SM-2059 (manufacturer: General Electric), SLM-55067 (manufacturer: Wacker), and Abil®-Quat 3270 and 3272 (manufacturer: Th. Goldschmidt; diquaternary polydimethylsiloxanes, Quaternium-80). A suitable anionic silicone oil is the product Dow Corning® 1784.

Additional active substances, auxiliary substances, and additives are, for example:
thickeners such as agar-agar, guar gum, alginates, xanthan gum, gum arabic, gum karaya, locust bean gum, linseed gums, dextranes, cellulose derivatives, e.g., methyl cellulose, hydroxyalkyl cellulose, and carboxymethyl cellulose, starch fractions and derivatives such as amylose, amylopectin, and dextrins, clays such as bentonite, or fully synthetic hydrocolloids such as polyvinyl alcohol,
hair-conditioning compounds such as phospholipids, for example soy lecithin, egg lecithin, and cephalins, and silicone oils,
perfume oils, dimethyl isosorbide, and cyclodextrins,
solvents and solubilizers such as ethanol, isopropanol, ethylene glycol, propylene glycol, glycerol, and diethylene glycol,
fiber-structure-improving active substances, particularly mono-, di-, and oligosaccharides, such as glucose, galactose, fructose, fruit sugar, and lactose,
conditioning active substances such as paraffin oils and plant oils, for example sunflower oil, orange oil, almond oil, wheat germ oil, and peach kernel oil,
quaternized amines such as methyl-1-alkylamidoethyl-2-alkylimidazolinium methosulfate,
defoamers such as silicones,
dyes for dyeing the agent,
anti-dandruff active substances such as piroctone olamine, Zinc Omadine, and climb azole,
active substances such as bisabolol,
cholesterol,
consistency regulators such as sugar esters, polyolesters, or polyol alkyl ethers,
fatty acid alkanolamides,
complexing agents such as EDTA, NTA, β-alanine diacetic acid, and phosphonic acids,
swelling and penetrating substances such as primary, secondary, and tertiary phosphates,
opacifiers such as latex, styrene/PVP copolymers, and styrene/acrylamide copolymers,
pearlizing agents such as ethylene glycol mono- and distearate and PEG-3 distearate,
pigments,
propellants such as propane-butane mixtures, N2O, dimethyl ether, CO2, and air,
antioxidants.

With regard to additional optional components and the used amounts of these components, the pertinent manuals known to a person skilled in the art are expressly referenced.

The method according to the invention for treating hair, in which a composition including at least one casein hydrolysate is applied to the hair and is rinsed from the hair after an exposure time, can be performed as follows:

a) applying a composition including a casein hydrolysate to the hair,
b) allowing the composition to act for a time period in the range from seconds to the time until the next hair wash,
c) optionally rinsing the composition out of the hair.

The exposure time is preferably approximately 5 seconds to 100 minutes, especially preferably 5 seconds to 50 minutes, and exceedingly preferably 5 seconds to 20 minutes.

A method in which a composition including at least one casein hydrolysate is applied to the hair and remains there is also in accordance with the invention. According to the invention, “to remain on the hair” is understood to mean that the agent is not rinsed out of the hair immediately after the use of the agent. Rather, in this case the agent remains on the hair for a time period in the range from more than 100 minutes to the time until the next hair wash.

The compositions including at least one casein hydrolysate that are used in the method according to the invention or the use according to the invention can be single-phase or multi-phase compositions, for example in particular can have two or three discrete phases that are visually clearly separated. If the compositions are multi-phase compositions, the entire composition is converted into a single-phase composition by shaking before use and then is used. After the shaking, spontaneous unmixing occurs and the discrete phases are formed again within approximately 1 minute to 300 minutes. If the composition is a multi-phase composition, the composition is packaged in optically transparent packaging.

In a preferred embodiment, the compositions are formulated as conditioning shampoos, conditioners, hair masks, hair packs, hair tonics, hair gels, hair waxes, or combinations thereof

For this purpose, it is advantageous if the entire composition has a viscosity of 1000 to 50000, more advantageously 3000 to 30000, more preferably 5000 to 25000, and most preferably 5000 to 20000 mPas. The viscosity is measured in accordance with methods well known to a person skilled in the art.

Of course, the compositions for the method according to the invention and the use according to the invention including at least one casein hydrolysate can be formulated as a pump spray, aerosol spray, pump foam, or aerosol foam.

For this purpose, the compositions are packaged in a dispensing device, which is either a compressed-gas container (“aerosol container”) additionally filled with a propellant or a non-aerosol container.

By definition, the compressed-gas containers used to distribute a product via a valve by means of the internal gas pressure of the container are called “aerosol containers”. Conversely to the aerosol definition, a container under normal pressure used to distribute a product by means of mechanical action by a pump system is defined as a “non-aerosol container”.

The agents according to the invention are preferably formulated as aerosol hair foams or aerosol hair sprays. The agent according to the invention therefore preferably additionally includes at least one propellant.

Propellants suitable according to the invention are selected, for example, from N₂O, dimethyl ether, CO2, air, alkanes having 3 to 5 carbon atoms, such as propane, n-butane, isobutane, n-pentane, and isopentane, and mixtures thereof. Dimethyl ether, propane, n-butane, isobutane, and mixtures thereof are preferred.

According to a preferred embodiment, the mentioned alkanes, mixtures of the mentioned alkanes, or mixtures of the mentioned alkanes with dimethyl ether are used as a single propellant. However, the invention expressly also comprises the additional use of propellants of the type of the chlorofluorocarbons, particularly the fluorocarbons.

For a given spraying device, the sizes of the aerosol droplets or of the foam bubbles and the size distribution can be set by means of the amount ratio of propellant to the other constituents of the preparations.

The amount of propellant used varies in accordance with the specific composition of the agent, the packaging used, and the desired product type, such as hair spray or hair foam. If conventional spraying devices are used, aerosol foam products include the propellant preferably in amounts of 1 to 35 wt %, with respect to the entire product. Amounts of 2 to 30 wt %, particularly 3 to 15 wt %, are especially preferred. Aerosol sprays generally include larger amounts of propellant. In this case, the propellant is preferably used in an amount of 30 to 98 wt %, with respect to the entire product. Amounts of 40 to 95 wt %, particularly 50 to 95 wt %, are especially preferred.

The aerosol products can be produced in a typical manner. In general, all constituents of the particular agent with the exception of the propellant are introduced into a suitable pressure-resistant container. The container is then closed with a valve. Finally, the desired amount of propellant is introduced by conventional techniques.

For the foaming of agents in the form of a gel in a two-chamber aerosol container, isopentane is preferably suitable as a propellant, which is incorporated into the agent according to the invention and is packaged in the first chamber of the two-chamber aerosol container. At least one additional propellant different from isopentane is packaged in the second chamber of the two-chamber aerosol container and builds up a higher pressure in the two-chamber aerosol container than the isopentane. The propellants of the second chamber are preferably selected from N₂O, dimethyl ether, CO₂, air, alkanes having 3 or 4 carbon atoms (such as propane, n-butane, and isobutane), and mixtures thereof

However, preferred compositions of the method according to the invention and of the use according to the invention are formulated as non-aerosols. For this purpose, specific pumps and conveying systems are necessary, as already stated. These are well known to a person skilled in the art. Known and excellently suitable systems are provided, for example, by Airspray International BV, for example under the product name Airfoamer.

In the case of the formulation as a non-aerosol foam in a suitable container having a suitable conveying and pumping mechanism, the foam is generally produced by a fine-mesh sieve in the pump head by means of air simultaneously sucked in by the pump. For this purpose, it is advantageous if the entire composition according to the invention has a viscosity of 1 to 35000, more advantageously 1 to 10,000, more preferably 1 to 5000, and most preferably 2 to 500 mPas. The viscosity is measured in accordance with methods well known to a person skilled in the art.

The statements made with regard to the agents according to the invention apply, mutatis mutandis, with regard to preferred embodiments of the methods according to the invention.

The following examples should illustrate the invention in more detail.

### EXAMPLES

Unless otherwise noted, all specifications are in parts by weight.

1. Proof of effectiveness

1.1 Increase in keratin synthesis

The hair structure is largely dependent on the composition of particular hair-specific structural proteins, the hair keratins.

Specific hair-keratin-regulating genes were selected on the basis of internationally renowned publications. Hair keratins form the quantitatively largest portion of the large number of hair-shaft-forming structural proteins and ensure the strength of the hair. The hair keratins are divided into two groups, type I keratins (acidic hair keratins) and type II keratins (basic to neutral hair keratins). Examinations have shown that certain hair keratins and cytokeratins decrease with increasing age. In particular, this pertains to the hair keratins KRT33A, KRT34, and KRT86. The gene expressions of these hair keratins were determined in the examinations. Therefore, this selection combines the predominant knowledge about the regulation of hair growth. On the basis of an expression profile of these genes, conclusions about hair structure and cell aging in vivo are possible. After substances that have effects on the biologically active part of the hair have been applied, the determination of the expression profiles of these genes provides statements about the strength and structure of the hair.

KGF is an important growth factor that is released by the dermal papilla in order to control the proliferation of the hair keratinocytes. This parameter is influenced in every case of a substance that potentially strengthens hair.

### Example 1: Proof of the Differential Expression of Hair-Relevant Genes

Hair keratins form the quantitatively largest portion of the large number of hair-shaft-forming structural proteins and ensure the strength of the hair.

The expression of various hair keratins in the organotypic model can be examined by means of a quantitative real-time PCR method. To perform the PCR, the RNA is first isolated from the organotypic models by means of the RNeasy Mini Kit from the company Qiagen and transcribed into cDNA by means of reverse transcription. In the subsequent PCR reaction, which is performed by means of gene-specific primers for the particular hair keratins and is used to amplify the desired gene segments, the formation of the PCR products is detected online by means of a fluorescent signal. The fluorescent signal is proportional to the amount of the formed PCR product. The stronger the expression of a certain gene is, the greater the amount of formed PCR product is and the higher the fluorescent signal is.

To quantify the gene expression, the untreated control is set to 1 and the expression of the genes to be determined is set in relation thereof (x-fold expression). Values that are greater than or equal to 1.5 times the expression of the untreated control are classified as significant.

TABLE 1
Influence of hydrolyzed milk protein
on the expression of hair-relevant genes
Expression scheme of various hair keratins after 24-h
treatment of hair follicle models with hydrolyzed milk
protein in relation to the untreated control [=1]

| | KRT33A | | KRT34 | | KRT86 | |
|---|---|---|---|---|---|---|
| | | Standard | | Standard | | Standard |
| | Mean | deviation | Mean | deviation | Mean | deviation |
| Control | 1.0 | 0.3 | 1.0 | 0.3 | 1.0 | 0.2 |
| Casein | 1.3 | 0.5 | 1.4 | 0.5 | 1.4 | 0.5 |
| hydrolysate | | | | | | |
| 0.005% | | | | | | |
| Casein | 1.3 | 0.2 | 1.1 | 0.5 | 1.0 | 0.3 |
| hydrolysate | | | | | | |
| 0.01% | | | | | | |
| Casein | 2.1 | 0.5 | 2.1 | 0.7 | 1.8 | 0.9 |
| hydrolysate | | | | | | |
| 0.05% | | | | | | |

Hydrolyzed milk protein leads to increased gene expression of various hair keratins. On the basis of this expression profile, the hair-structure-promoting effect of hydrolyzed milk protein can be deduced.

### Example 2: Proof of the Release of Growth Factors

The keratinocyte growth factor (KGF) is an important regulator of hair growth that is released by the dermal papilla and switches on additional biological processes at various points in the hair follicle. In the case of a potentially growth-promoting and hair-strengthening substance, an increase in the factor released into the medium can be assumed. Specifically, KGF stimulates the proliferation of keratinocytes. Accordingly, KGF is more greatly expressed in the growth phase. For this purpose, the release of KGF is quantified by means of a hair follicle model. The examination of the release of the growth factor into the medium occurred after a treatment time of 72 h. Untreated models were carried along as a control. All examinations occurred in triplicate. The release was determined by means of the Bio-Plex method. The Bio-Plex examination is based on the principle of a sandwich immunoassay. A specific, bead-conjugated primary antibody binds to the target protein in the sample, which target protein in turn is specifically detected by a fluorescence-marked detectio antibody.

TABLE 2
Increase in KGF release after 72-h treatment of the organotypic
hair follicle model in comparison with untreated controls.

KGF (%)

| 72 h | Mean | Standard deviation |
|---|---|---|
| Control | 100 | 7 |
| Casein hydrolysate 0.005% | 129 | 11 |
| Casein hydrolysate 0.01% | 197 | 17 |
| Casein hydrolysate 0.05% | 164 | 5 |

The analyses of the hair follicle model resulted in a concentration-dependent increase in the release of KGF by at most +97% (table 2). The growth and strength of healthy and vital hair are excited by the stimulation of KGF.

### 2. Additional Formulation Examples

a) Hair gel:

| | |
|---|---|
| Cetyl/stearyl alcohol + 30 EO | 19.0 |
| Oleyl alcohol + 10 EO | 8.0 |
| Cetiol ® HE¹ | 15.0 |
| Cetiol ® LC² | 4.0 |
| Casein hydrolysate | 4.0 |
| Tocopheryl acetate | 0.4 |
| Panthenol | 2.0 |
| Glycerol | 4.0 |
| Perfume oil | 0.6 |
| Water | ad 100 |

¹Polyol fatty acid ester (CTFA name: PEG-7-Glyceryl Cocoate) (HENKEL)
²Caprylic/capric acid esters with saturated fatty alcohols C12-C18 (CTFA name: Coco-Caprylate/Caprate) (HENKEL)

b) Shampoo

| | |
|---|---|
| Sodium lauryl (2-EO) sulfate | 12.0 |
| Coco-betaine | 5.0 |
| Casein hydrolysate | 1.0 |
| Piroctone olamine | 0.8 |
| Plant-based protein hydrolysate | 0.5 |
| Perfume oil | 0.8 |
| Water | ad 100 |

Hair Masks:

| | K1 | K2 | K3 | K4 | K5 | K6 | K7 | K8 | K9 | K10 |
|---|---|---|---|---|---|---|---|---|---|---|
| Casein hydrolysate | 0.5 | 0.2 | 0.8 | 0.3 | 0.1 | 0.05 | 0.1 | 0.2 | 0.3 | 0.4 |
| Quaternium-98 | 1.0 | 1.5 | 2.0 | 2.5 | 3.0 | 2.0 | 2.5 | 3.0 | 1.5 | 1.0 |
| Distearoylethyl | 2.0 | 0.5 | 1.0 | 1.0 | 1.0 | 2.0 | 0.5 | 1.0 | 1.0 | 1.0 |
| hydroxy- | | | | | | | | | | |
| ethylmonium | | | | | | | | | | |
| methosulfate | | | | | | | | | | |
| Stearamidopropyl | 0.5 | 1.0 | 1.0 | 1.5 | 1.0 | 0.5 | 1.0 | 1.0 | 1.5 | 1.0 |
| dimethylamine | | | | | | | | | | |
| Behentrimonium | 0.5 | 0.5 | — | — | — | — | 0.5 | — | — | — |
| chloride | | | | | | | | | | |
| Polyquaternium-37 | 0.5 | 0.3 | 0.5 | — | 0.5 | — | — | 0.3 | — | 0.3 |
| Keradyne ® HH | — | 1.5 | — | — | — | 1.0 | — | — | — | — |
| Quaternium-27 | 1.5 | — | 1.0 | — | — | — | 1.0 | | 0.7 | 1.5 |
| Polyquaternium-77 | — | 0.5 | 0.5 | 0.3 | — | 0.3 | 0.5 | — | 0.3 | 0.3 |
| Polyquaternium-16 | 0.3 | — | — | — | 0.5 | — | — | 0.3 | — | — |
| Glycerol | 0.5 | 1.0 | 1.5 | 2.0 | 3.0 | 4.0 | 0.5 | 1.0 | 1.5 | 2.0 |
| Isopropyl myristate | 0.2 | 0.5 | 0.5 | 1.0 | 0.5 | 0.2 | 0.5 | 0.5 | 1.0 | 0.5 |
| Shea butter | — | — | — | — | — | 0.5 | — | — | — | — |
| Oleyl oleate | 0.4 | 0.3 | — | — | — | 0.3 | — | — | — | — |
| Cetiol ® OE | 0.2 | — | 0.3 | — | 0.2 | — | — | — | — | — |
| Dicaprylyl | — | 0.2 | — | 0.2 | — | 0.5 | 0.3 | — | — | — |
| carbonate | | | | | | | | | | |
| Glyceryl | 0.3 | 0.3 | 0.3 | 0.3 | 0.3 | 0.3 | 0.3 | 0.3 | 0.3 | 0.3 |
| monostearate | | | | | | | | | | |
| Cetearyl alcohol | 3.0 | 3.0 | 3.0 | 3.0 | 3.0 | 3.0 | 3.0 | 3.0 | 3.0 | 3.0 |
| Hydrolyzed keratin | 0.3 | — | — | — | — | — | — | — | — | — |
| Taurine | — | 0.1 | — | — | — | — | — | — | — | — |
| Carnitine | — | — | 0.1 | — | — | — | — | — | — | — |
| Crodarom ® Pearl | — | — | — | 0.2 | — | — | — | — | — | — |
| Glycine | — | — | — | — | 0.3 | — | — | — | — | — |
| Coenzyme Q₁₀ | — | — | — | — | — | 0.05 | — | — | — | — |
| Ectoine | — | — | — | — | — | — | 0.1 | — | — | — |
| Echinacea extract | — | 0.3 | — | — | — | — | — | 0.5 | — | — |
| Apricot kernel oil | 0.2 | — | — | 0.2 | — | — | 0.3 | — | 0.1 | 0.2 |
| Dimethicone | — | — | — | — | — | — | — | — | 0.5 | — |
| 60,000 cSt | | | | | | | | | | |
| Dimethiconol | — | — | — | — | — | — | — | — | — | 0.5 |
| 1000000 cSt | | | | | | | | | | |
| Amodimethicone, | 0.5 | — | — | — | — | 0.5 | — | — | — | — |
| e.g., Dow | | | | | | | | | | |
| Corning ® 959 | | | | | | | | | | |
| Silicone | — | — | — | 0.3 | — | — | — | 0.3 | — | — |
| Quaternium-22 | | | | | | | | | | |
| Wacker | — | — | 0.5 | — | — | — | 0.3 | — | — | — |
| Belsil ® ADM | | | | | | | | | | |
| 8301E | | | | | | | | | | |
| Climbazole | — | 0.3 | — | — | — | — | — | — | — | — |
| Octopirox | — | — | — | — | — | 0.5 | | — | — | — |
| Lauryl glucoside | 0.5 | — | 0.5 | — | 0.5 | — | 0.5 | — | 0.5 | — |
| Trehalose | — | 0.8 | — | — | — | — | — | — | — | — |
| Glucose | — | — | — | 0.5 | — | — | — | 0.5 | — | — |
| Panthenol | — | 0.5 | — | 0.5 | — | 0.5 | — | 0.5 | — | 0.5 |
| Niacin | 0.3 | — | 0.3 | 0.5 | — | 0.3 | — | 0.5 | 0.3 | — |
| Benzophenone-4 | 0.3 | — | — | 0.5 | — | 0.2 | — | — | — | 0.1 |

| | |
|---|---|
| Citric acid | For setting the pH value to 2.0 to 4.5 |
| Preservative, | Water ad 100 |
| perfume | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic method for improving hair growth, hair structure, and the strength, resistance, and shine of the hair, comprising treating the hair with a composition including at least one casein hydrolysate and common carrier substances, active substances, and auxiliary substances.

2. The cosmetic method according to claim 1, wherein the composition includes 0.0001 to 5 wt % of casein hydrolysate, based on the total weight of the composition.

3. The cosmetic method according to claim 1, wherein the composition includes 0.005 wt % to 3.0 wt % of casein hydrolysate, based on the total weight of the composition.

4. The cosmetic method according to claim 1, wherein the composition includes 0.005 to 1 wt % of casein hydrolysate, based on the total weight of the composition.

5. The cosmetic method according to claim 1, wherein the casein hydrolysate has a molar mass of 500 to 8000 daltons.

6. The cosmetic method according to claim 1, wherein the casein hydrolysate has a molar mass of 800 to 8000 daltons.

7. The cosmetic method according to claim 1, wherein the casein hydrolysate has a molar mass of 1000 to 5000 daltons.

8. The cosmetic method according to one of claim 1, wherein the casein hydrolysate is produced from κ-casein and/or lactophorin.

9. The cosmetic method according to claim 1, wherein the composition additionally includes at least one carnitine derivative selected from the group consisting of acetyl-L-carnitine, L-carnitine fumarate, L-carnitine citrate, lauroyl-L-carnitine, L-carnitine tartrate.

10. The cosmetic method according to claim 1, wherein the composition further comprises taurine (2-aminoethanesulfonic acid).

11. The cosmetic method according to claim 1, wherein the composition further comprises N—N-monomethyltaurine and/or N,N-dimethyltaurine.

12. The cosmetic method according to claim 1, wherein the composition further comprises at least one plant extract.

13. The cosmetic method according claim 1, wherein the composition further comprises 0.0001 to 10 wt %, with respect to the entire agent, at least one vitamin or one vitamin precursor.

14. The cosmetic method according to claim 1, wherein the composition further comprises at least one purine.

15. The cosmetic method according to claim 1, wherein the composition includes at least one additional protein hydrolysate and/or oligopeptide different from the casein hydrolysate.
